(19) United States
(12) Patent Application Publication
(10) Pub. No.: US 2010/0203634 A1
(43) Pub. Date: Aug. 12, 2010

(54) Inhibition of stem cell differentiation, enhancement of proliferation and selective induction of apoptosis by Wnt factors

(75) Inventor: Menno Frederik Kielman, Voorschoten (NL)

Correspondence Address: TRASKBRITT, P.C., P.O. BOX 2550, SALT LAKE CITY, UT, 84110, US

(73) Assignee: DeltaCell B.V., Leiden (NL)

(21) Appl. No.: 12/661,687

(22) Filed: Mar. 19, 2010

Related U.S. Application Data
(63) Continuation of application No. 11/432,775, filed on May 11, 2006, now abandoned.
Application No. 11/432,775 is a continuation of application No. PCT/NL2004/000789, filed on Nov. 11, 2004.
(60) Provisional application No. 60/519,403, filed on Nov. 11, 2003.

(30) Foreign Application Priority Data
Nov. 11, 2003 (EP) 03078549.7

(51) Int. Cl. C12N 5/074

(52) U.S. Cl. 435/366; 435/377

(57) ABSTRACT

The invention relates to a method for at least in part inhibiting differentiation of stem cells in a population of mammalian cells comprising up-regulating a Wnt-signaling pathway to a differentiation-inhibiting level in the population of cells. The invention increases at the same time the number of stem cells in a population of mammalian cells compared to a reference population, and induces, at least in part, apoptosis in mesenchymal cells in a population of mammalian cells. The invention also discloses a method for selective differentiation of a stem cell, comprising controlling the level of Wnt pathway activation. The invention is used for the proliferation and subsequent differentiation of embryonic stem cells and lung stem cells, and for ex vivo lung explant cultivation.

### CROSS-REFERENCE TO RELATED APPLICATIONS

This application is a continuation of U.S. patent application Ser. No. 11/432,775, filed May 11, 2006, pending, which is a continuation of PCT International Patent Application PCT/NL2004/000789, filed Nov. 11, 2004, designating the United States of America, and published, in English, as PCT International Patent Publication No. WO 2005/052141 A1 on Jun. 9, 2005, which claims the benefit of European Patent Application No. 03078549.7 filed Nov. 11, 2003, and U.S. Provisional Patent Application Ser. No. 60/519,403 filed Nov. 11, 2003, the entire contents of each of which are hereby incorporated herein by this reference. This application also claims the benefit, under 35 U.S.C. §119(e), of the aforementioned U.S. Provisional Patent Application Ser. No. 60/519,403 filed Nov. 11, 2003.

### TECHNICAL FIELD

The invention relates generally to the fields of biotechnology and medicine, in particular, to the field of pulmonary medicine. The invention relates to the differentiation of stem cells, in particular, to organ tissue regeneration and recovery and to transplantation. The invention, more in particular, relates to inhibition of stem cell differentiation by manipulating a Wnt pathway. More in particular, the invention relates to enhancing proliferation and controlling the differentiation of lung cells by manipulating a Wnt pathway.

### BACKGROUND

Vital organs like the lung, the kidney, the liver, the pancreas, and the skin are characterized by the presence of organ-specific differentiated epithelial cells based on an organ-specific matrix of connective tissue (mesenchymal) cells. The combination of the differentiated cells with the connective tissue matrix is related to the specific function of each such organ. The specific functions may be as varied as, for example, gas exchange in the lung, filtration in the kidney, detoxification and conjugation in the liver, insulin production in the pancreatic islet cells or protection against a hazardous environment by the skin. Disease or degeneration of such an organ is often life threatening because degenerated or lost organ structure is often poorly replaced and because the specialized cells of one organ cannot take over the function of another organ.

Worldwide, solutions have been sought for the treatment of degenerative disease of organs and the skin and its appendices. For the skin, as a relatively simple organ structure, some advances have been made by application of autologous or heterologous skin transplants. Pieces of skin are treated (meshed) in order to be stretched out beyond their former size and cover the skin defects of a patient with meshed skin. Such transplants offer a basis for cells to proliferate and close the skin lesions.

For the specialized organs in the body, regenerating cells within the organ in situ would be most optimal because transplantation is often more difficult and less successful. Transplantation often leads to adverse effects of host versus graft or even graft versus host. It would also be a major achievement if organs or a part thereof, could be kept outside of the body (syn. ex vivo) for a period of time, to give them a chance to recover by up-regulating the proliferation and differentiation of the organ-specific cells. While the organ is ex vivo, the patient is, for instance, dependent of machines to take over the organ's function. In a later stage, the organ is placed back in the patient. This method is also very suitable for bone marrow cells that are taken out of the patient before treatment of leukemia and are replaced after the treatment. The bone marrow cells are often separated in various subpopulations and tested for the absence of leukemic cells to avoid the leukemic cells from re-entering the host. Many cells do not survive this ex vivo handling, with the result that re-implantation of the bone marrow is less successful.

Highly differentiated cells like, for example, the kidney cells, the insulin-producing cells in the Islets of Langerhans of the pancreas, and glandular and/or hair follicle cells of the dermis, are especially very difficult to recover, if possible at all, and very difficult to maintain once taken out of their context in the body.

Many researchers try to find ways of extending the survival time of these specialized cells in culture and possibilities of proliferating the cells while retaining their differentiated state. In practice, it appears to be hard to reach this goal.

In the present invention, this objective is reached by enhancement of the Wnt-signaling pathway in stem cell/progenitor cells of such an organ comprising differentiated cells. In one embodiment, an extended survival time and proliferation, differentiation, and selective enrichment of stem cells of the lung are disclosed.

One of the main potential applications of stem cells is their use in transplantation studies in which these cells reconstitute the cells in the diseased organ. Transplantation is preferably performed with stem cells that can either differentiate in vivo to the desired cell type or with in vitro pre-differentiated stem cells. Pre-differentiation of stem cells in vitro to a desired cell type is a process that was difficult to control before this invention.

One aspect of the present invention provides a method to direct the differentiation of stem cells to a desired cell type in vitro in order to use the differentiated cells for transplantation purposes (see, for instance, Example I). Control of differentiation is achieved by limiting the enhancement of the Wnt-signaling pathway to a certain window in these stem cells. Control of the Wnt-signaling pathway is, for instance, achieved by the addition of at least one component of the Wnt-signaling pathway and/or at least one affector of gene expression that is capable of interfering with the Wnt-signaling pathway. Without being bound by theory, it is thought that the fluctuation of the concentration of intracellular beta catenin is kept within a narrow range, thereby promoting the differentiation of a limited number of cell types.

The Wnt gene family encodes developmentally important secreted factors involved in cell growth, differentiation and organogenesis (Wodartz and Nusse, 1998). Wnt genes encode a family of secreted glycoproteins that modulate cell fate and behavior in embryos through activation of receptor-mediated signaling pathways. Wnt-signaling events are initiated by receptor activation involving binding to the cysteine-rich domain (CRD) of frizzled seven-transmembrane receptor protein (Fz) (Bhanot et al., 1996). A classical Wnt signal suppresses the activity of glycogen synthase kinase 3 (GSK-3), leading to changes in phosphorylation and increased stability of the β-catenin protein in the cytoplasm (Hinck et al., 1994). β-catenin is essential for activating target genes in response to Wnt signaling (Miller and Moon, 1996; Willert and Nusse, 1998), since it complexes with HMG box transcription factors of the TCF/LEF family (Behrens et al., 1996; Molenaar et al., 1996; Huber et al., 1996). Wnt sequences, patterns of expression and activities are highly conserved in evolution, so it has been possible to gain insights into the functions and mechanisms of action of the Wnt genes through a synthesis of genetic and cell biological approaches in different organisms. These studies suggest that there are functionally distinct WNT proteins as assayed by the ability to transform cells and by differences in embryonic responses to ectopic WNT signals. Moreover, gain-of-function and loss-of-function studies both support the involvement of Wnt proteins in modulating cell fate and cell behavior during vertebrate development, often through combinatorial interactions with other signaling pathways to regulate gene expression (R. T. Moon, J. D. Brown, M. Torres, Trends Genet. 1997 April; 13(4):157-62). This is supported by data on the ability and sensitivity of mouse embryonic cells to differentiate into three germ layers, which was inhibited by specific mutations in the adenomatous polyposis coli gene (Kielman et al., 2002).

Components of the Wnt-signaling pathway have been found to be present during organogenesis in the mouse (Roelink and Nusse, 1991; Buhler et al., 1993; Parr et al., 1993; Christiansen et al., 1995; Wang and Shackleford, 1996; Cho and Dressler, 1998; Korinek et al., 1998; Leimester et al., 1998). Moreover, loss of function of Wnt and Wnt-related genes leads to abnormal development in the mouse (McMahon and Bradley, 1990; Monkley et al., 1993; Takada et al., 1994; Stark et al., 1994; Galceran et al., 1999; Liu et al., 1999; Yamaguchi et al., 1999; Brisken et al., 2000; Lee et al., 2000). Wnt signaling is inhibited by the presence of Dickkopf proteins. Dickkopf (Dkk) proteins bind to the LRP co-receptor for Wnt. Modulating the Wnt pathway in cell cultures can, for instance, be performed by genetic modification (Kielman et al., 2002) or by administering soluble factors influencing the Wnt pathway. The latter method has been used for monolayers of cells in cell culture, wherein the cells had no tissue connection to each other like, for example, hemopoietic stem cells (Matthews et al., 2000). In organ culture, the effects of soluble factors is not easily predicted or understood because of the three-dimensional structure of the organ and because the microenvironment of the cells is more complex by the presence of other cells such as connective tissue and blood vessels.

### SUMMARY OF THE INVENTION

The present invention provides the controlling of the canonical Wnt-signaling pathway and discloses in one embodiment Wnt3a functioning as a protein of the canonical Wnt pathway. In one aspect of the present invention, it is demonstrated that Wnt3a is capable of inhibiting the proteolytic breakdown of intracellular beta-catenin, thereby inhibiting and/or modulating the differentiation and replication of pluripotent stem cells in the lung and of omnipotent stem cells.

In one embodiment, the present invention provides the use of a factor that enhances the canonical Wnt-signaling pathway to levels that prevent, inhibit, or modulate, or at least retard, the differentiation of stem cells from the lung and/or of omnipotent stem cells and enhance their proliferation.

The concentration of factor needed to enhance the Wnt-signaling pathway in order to inhibit differentiation and/or to enhance proliferation, is depending on the stem cell type and the relevant factor. As an example, the effective concentration needed for the proliferation of lung cells is disclosed in the examples, but with the method of the invention, a skilled person is taught how to find the effective concentration for other cell types and organ types. For recombinant human Wnt3a as produced by R&D Systems (cat nr. 1323-WN), the effective concentration for lung stem cells is between 1 and 2000 ng/ml. Therefore, the invention discloses a method for at least in part inhibiting differentiation of stem cells in a population of mammalian cells comprising up-regulating a Wnt-signaling pathway to a differentiation-inhibiting level in the population of cells. Preferably, up-regulating the Wnt-signaling pathway is achieved by contacting the cells with a soluble Wnt3a factor.

Nowadays it is possible by differential adhesion and percoll gradient centrifugation techniques to isolate a stem cell population from lung and airway tissue. Long-term culture (for example, for more than four or five days) of these stem cells without losing the stem cell properties was not possible before this invention.

One embodiment of the present invention provides a method to expand the total number of somatic stem cells present in a population of cells such as, for example, lung and airway tissue (including the tracheal compartment, the bronchial compartment, the bronchiolar compartment and the alveolar compartment) and of omnipotent stem cells (such as embryonic stem cells) in vitro, while at least in part preserving their original stem cell properties. The invention provides a method for increasing the number of stem cells in a population of mammalian cells compared to a reference population, comprising up-regulating a Wnt-signaling pathway in the population of cells to a differentiation-inhibiting level, wherein the reference population is not provided with up-regulating of a Wnt-signaling pathway.

In one embodiment of this invention, stem cells are isolated from healthy lung tissue and expanded in vitro and used as a source of stem cells that are transplanted directly to a patient in order to generate new lung or airway tissue.

The invention now also enables a skilled person to isolate lung or airway stem cells from a lung sample of the patient's lung or airway tissue and to expand these patient-derived stem cells in sufficient numbers in vitro in order to return these stem cells to the patient. Therefore, the invention provides a method for selective differentiation of a stem cell, comprising controlling the level of Wnt pathway activation.

In another embodiment, the invention provides genetic modification of the cells of a person with a genetic defect in the lung cells, and it provides a method for isolating, culturing and expanding a patient's own stem cells in vitro and to modify them genetically in order to correct the effect of this genetic defect present in the patient's germ line and to return these genetically modified stem cells to the patient. Therefore, the present invention provides a cell obtained by a method of the invention for selective differentiation comprising controlling a Wnt-signaling pathway.

In another embodiment, because with the present invention stem cells are cultured for a longer period of time, cultured stem cells from lung and airway tissue are used as a source to generate differentiated lung and airway tissue in vitro in order to transplant these to a patient with a lung or airway defect. Therefore, in another embodiment, this application provides a method for repopulating cells in a mammal comprising administering to the mammal a therapeutically effective amount of a stem cell or a lung stem cell of the invention. This method is particularly useful for the treatment of people suffering from or susceptible to lung failure, the lung failure, for example, caused by lung surfactant deficiency, emphysema, chronic respiratory distress, and/or cancer. The cells are in one embodiment taken out of the body to be treated according to the present invention, or one chooses to treat the cells in the body. Therefore, the present invention discloses a method for, at least in part, inhibiting differentiation of stem cells, and/or increasing the numbers of stem cells, and/or enhancing the apoptosis of mesenchymal cells, and/or differentiating the stem cells in the mammalian body, preferably in a human body, comprising manipulating the Wnt pathway. In another embodiment, the invention provides a method to prevent or retard the differentiation of omnipotent stem cells. Such omnipotent stem cells have the ability to differentiate into all the possible cell types and cell lineages that are present in the full-grown organism. Omnipotent stem cells are preferably isolated from the inner-cell mass of early embryos at the blastocyst stage. The invention is also valid for omnipotent stem cells that can be isolated from early embryos at other developmental stages than the blastocyst stage. The invention is also suitable for omnipotent stem cells that are isolated from the genital ridge, the so called primordial germ cells (PGCs), of early embryos that already underwent the formation of the three germ layers: ectoderm, endoderm and mesoderm during gastrulation. The invention is also suitable to omnipotent stem cells that are obtained by the transfer of nuclei from differentiated somatic cells (preferably from a patient) to one of the omnipotent stem cells described in this patent.

One of the applications in which omnipotent stem cells are used is the generation of transgenic animals by transfecting an omnipotent cell with a DNA-construct in such a way that the original DNA content of that cell is changed. Such a DNA construct is maintained in the cell by an interchromosomal integration (nuclear or mitochondrial) or as an episomal (extra-nuclear) DNA molecule. Maintenance of the omnipotent cells is facilitated by the addition of leukocyte inhibitory factor or other growth factors, preferably added in a purified form or produced by a cell-line. The present invention provides an alternative to these growth factors as addition of a Wnt pathway-elevating factor at an effective concentration helps to maintain the omnipotent characteristics of the stem cells.

Another embodiment provides a method for the generation and isolation of omnipotent stem cells from the embryo. Omnipotent stem cells are isolated from the embryo at different developmental stages. The difficulty in this process is the loss of the omnipotent properties of the stem cells due to differentiation into various cell types. This invention at least prevents or retards the differentiation of these omnipotent stem cells, thereby facilitating the isolation of omnipotent stem cell lines from embryos, while maintaining their omnipotent properties. Before this invention, isolation of omnipotent stem cells from early embryos was very difficult, if possible at all, in many species including humans. This invention now allows for isolation of omnipotent stem cells from various mammalian species including humans with a much higher frequency and efficiency than was previously possible.

In another embodiment, the invention provides the insight that the induction of apoptosis by activation of the Wnt pathway is possible in every cell type present in the lung, i.e., terminally differentiated cells as well as stem cells. The concentration of a factor needed to induce apoptosis in a specific cell type is dependent on the factor itself and on the cell type. This concentration is for cell types present in the lung disclosed in Example 1, FIGS. 2 and 3. The level of Wnt polypeptide needed for apoptosis of mesenchymal cells is close to the level needed for inhibition of the differentiation and the enhancement of stem cell proliferation. Therefore, the present application discloses a method for inducing apoptosis in mesenchymal cells in a population of mammalian cells comprising up-regulating a Wnt-signaling pathway in the population of cells to a differentiation-inhibiting level. Preferably, the population also comprises a stem cell.

Apoptosis of mesenchymal cells prevents overgrowth of the somatic stem cells during their culture in vitro, thereby facilitating the purification and selective culture of lung stem cells in vitro.

Differentiation of omni- or pluripotent stem cells is dependent on the strength of the Wnt-signaling pathway in these cells. Stem cells in which the Wnt-signaling pathway is activated are limited in their differentiation capacity by the action of the Wnt signal. A stem cell in which the possible differentiating capacity or amplitude of the Wnt-signaling pathway is maximal can differentiate to all its derivatives. A stem cell in which the amplitude of the Wnt-signaling pathway is limited to a certain window is limited in its differentiation capacity and is restricted to differentiate to certain cell types.

This invention provides a method to direct the differentiation of omnipotent and pluripotent stem cells to a limited number of desired cell types, such as, for example, bronchial epithelial cells, tracheal epithelial cells, or epithelial alveolar type-I or II cells, by restricting the Wnt signal strength to a certain window. The present invention further provides a differentiated cell obtained by a method of the invention for selective differentiation of a stem cell, the method comprising controlling the level of Wnt pathway activation. As already described before, the present invention teaches how to direct differentiation in an organ or organ culture in vitro. Therefore, the present invention provides a method of the invention, wherein the population of cells is arranged in a three-dimensional structure. This invention further provides a soluble compound capable of influencing the propagation or the selected differentiation of a stem cell. The soluble compound, for example, comprises a soluble factor capable of up-regulating a Wnt-signaling pathway in the cells, such as, for example, an isolated Wnt factor or an antibody or a monoclonal antibody, capable of specifically binding and acting like a Wnt factor.

Therefore, the present inventions discloses a method of the invention, wherein the factor comprises a soluble factor capable of up-regulating a Wnt-signaling pathway in the cells and/or a soluble isolated Wnt factor, and/or an antibody and/or a monoclonal antibody with Wnt factor-like activity. In a preferred embodiment of the invention, the factor comprises Wnt3a or a functional part, derivative or analogue thereof. Therefore, the present invention also discloses a method of the invention, wherein the factor comprises Wnt3a or a functional part, derivative or analogue thereof.

The present invention discloses a range of concentrations at which a soluble Wnt factor, for instance, Wnt3a, is effective in inhibiting at least partial differentiation of stem cells in a population. Therefore, the present invention includes a method for inhibiting differentiation of a stem cell, preferably a lung stem cell, comprising providing the stem cell in vitro with a Wnt pathway-up-regulating substance.

The present invention also provides the insight that the stem cells are stimulated to proliferate by the presence of the soluble Wnt factor. Therefore, the invention also teaches a method for inducing a stem cell, preferably a lung stem cell, to proliferate by inhibiting differentiation of the stem cell, comprising providing the stem cell in vitro with a Wnt pathway-up-regulating substance. In one embodiment, the invention provides a method of the invention, wherein the Wnt pathway-up-regulating substance comprises a soluble Wnt3a factor or a functional part, derivative or analogue thereof. The invention furthermore disclose in the examples that Wnt3a already up-regulates a Wnt pathway from 1 ng/ml tissue culture fluid and is still up-regulating at 2000 ng/ml. Therefore, the invention provides a method of the invention, wherein the stem cell is provided with Wnt3a factor or a functional part, derivative or analogue thereof, in an amount of at least 1 ng and at most 2000 ng per ml tissue culture fluid. It is in the examples that the effects of Wnt3a on a Wnt pathway vary with the concentration of Wnt3a. Therefore, in another preferred embodiment, the invention teaches the above-described method, wherein the amount of Wnt3a factor or a functional part, derivative or analogue thereof, is from at least ten to at most 1500 ng per ml tissue culture fluid. The amount of Wnt3a factor or a functional part, derivative or analogue thereof, is more preferable from at least 20 to at most 1000 ng per ml tissue culture fluid, most preferably from at least 30 to at most 500 ng per ml tissue culture fluid.

In another embodiment, the present invention provides the insight that at a certain level of Wnt up-regulation, apoptosis in mesenchymal cells occurs while epithelial cells are not affected. This characteristic of Wnt up-regulation is used to selectively remove cells from a population of cells by choosing the level of Wnt up-regulation, such that apoptosis in mesenchymal cells is induced while epithelial cells are not affected. Therefore, the present invention provides a method for enriching a population of stem cells in a population of mesenchymal cells comprising the stem cells, by inducing apoptosis in mesenchymal cells by providing the mesenchymal cells with a Wnt pathway-up-regulating substance.

The present invention also provides the above-described method, wherein the population of stem cells comprises lung stem cells.

In another embodiment, the present invention teaches a method of the invention, wherein the Wnt pathway-up-regulating substance comprises a soluble Wnt3a factor or a functional part, derivative or analogue thereof.

Because the level of up-regulation of the Wnt pathway is dependent on the amount of Wnt3a administered to the cells, the invention also provides a method of the invention, wherein the mesenchymal stem cell is provided with Wnt3a factor or a functional part, derivative or analogue thereof, in an amount of at least 50 ng and at most 1500 ng per ml tissue culture fluid, such that mesenchymal cells are becoming apoptotic.

The examples disclose that the level of apoptosis is changed by changing the level of Wnt pathway up-regulation. Therefore, in a more preferred embodiment of the invention, a method of the invention is disclosed, wherein the mesenchymal stem cell is provided with Wnt3a factor or a functional part, derivative or analogue thereof, in an amount of at least 100 ng and at most 1000 ng per ml tissue culture fluid.

In another embodiment of the invention, the present invention also provides selective differentiation of stem cells that is dependent on the up-regulation of the Wnt pathway. Therefore, the present invention also provides a method for providing a plurality of stem cells from a population of mesenchymal cells and stem cells, comprising providing the mesenchymal cells with an apoptosis-inducing amount of a Wnt pathway-up-regulating substance and providing the stem cells with a differentiation-inhibiting amount of a Wnt pathway-up-regulating substance.

The above-described method is very suitable for lung stem cells. Therefore, a preferred embodiment of the invention provides a method of the invention, wherein the stem cells are lung stem cells.

The present invention provides the insight that Wnt3a is very suitable for up-regulating the Wnt pathway. Therefore, the present invention provides a method of the invention, wherein the Wnt pathway-up-regulating substance comprises a soluble Wnt3a factor or a functional part, derivative or analogue thereof.

An amount of Wnt3a between 50 ng and 1500 ng is preferred in order to achieve optimal results. Therefore, the present invention provides a method of the invention, wherein the soluble Wnt3a factor or a functional part, derivative or analogue thereof, is provided in an amount of at least 50 ng and at most 1500 ng per nil tissue culture fluid.

In a more preferred embodiment, the invention provides a method of the invention, wherein differentiation of a distal lung cell type is at least partly inhibited by administering 10 to 250 ng Wnt3a or a functional part, derivative or analogue thereof per ml tissue culture fluid.

The distal lung cell type is preferably an alveolar-type cell, more preferably, a type-I or type-II alveolar-type cell. Therefore, the present invention provides a method of the invention, wherein the distal lung cell type is a type-I or type-II alveolar cell.

Stem cells are capable of differentiating into various types of cells originating from a stem cell present in a population of lung stem cells. Up-regulation of the Wnt pathway influences that differentiation as is disclosed in the examples. Therefore, the present invention also provides a method of the invention, wherein proliferation and differentiation of an upper airway cell type is selectively increased by administering 10 to 750 ng Wnt3a or a functional part, derivative or analogue thereof per ml tissue culture fluid. In a preferred embodiment, the upper airway cell type is a tracheal or bronchial epithelial cell. Therefore, the present invention also provides a method of the invention, wherein the upper airway cell type is a tracheal and/or bronchial epithelial cell.

The present invention also provides a differentiated cell obtained by a method of the invention. The differentiated cell is suitable for providing to a person in need of new lung cells as a transplant.

Wnt3a is a soluble compound that up-regulates the Wnt pathway in cells. Such a compound is preferably included in a cell culture medium. Therefore, the present invention provides a kit comprising a cell culture medium and a component capable of influencing the Wnt-signaling pathway. This kit is useful for several purposes like, for instance, the proliferation of stem cells, and/or the differentiation of stem cells, and it may also be useful for the cultivation and differentiation of other cells than stem cells. The kit is also suitable for selectively depleting mesenchymal cells from a population of cells. Therefore, the present invention discloses a kit comprising a stem cell culture medium and a soluble Wnt3a factor or a functional part, derivative or analogue thereof in an amount of at least one to at most 2000 ng per ml cell culture medium.

### BRIEF DESCRIPTION OF THE FIGURES

FIG. 1, Panels A-H: Lung explant culture and subsequent immunohistological analysis after five days, without the addition (FIG. 1, Panels A-D, I and J) and with the addition of Wnt3a (1000 ng/ml, FIG. 1, Panels E-H, K and L). Note the arrest of alveolar differentiation, the primordial character of the epithelium and the massive induction of apoptosis in the mesenchyme in the presence of Wnt3a five days after the start of the culture.

FIG. 1, Panels I-L: Immunohistochemical analysis of employing PCNA as a marker for proliferation. FIG. 1, Panels I and J: PCNA staining five days after the start of the culture without the addition of Wnt3a. FIG. 1, Panels K and L: PCNA staining after five days of culture in the presence of Wnt3a (1000 ng/ml). Note the strong reduction in proliferation of the mesenchyme versus the epithelium in the presence of Wnt3a.

FIG. 2, Panels A-R: Lung explant culture and subsequent histological analysis two days after the start of the culture in the presence of Wnt3a at a concentration of 0 ng/ml (Panels A-C); 500 ng/ml (Panels D-F); 750 ng/ml (Panels G-I); 1000 ng/ml (Panels J-L); 2000 ng/ml (Panels M-O) and 4000 ng/ml (Panels P-R). Note the increasing number of apoptotic cells in the mesenchyme above 500 ng/ml Wnt3a and the induction of apoptosis in the epithelium above 2000 ng/ml Wnt3a.

FIG. 2, Panels S-AD: Immunohistochemical analysis of employing PCNA as a marker for proliferation two days after the start of the culture in the presence of Wnt3a at a concentration of 0 ng/ml (Panels S and T); 500 ng/ml (Panels U and V); 750 ng/ml (Panels W and X); 1000 ng/ml (Panels Y and Z); 2000 ng/ml (Panels AA and AB) and 4000 ng/ml (Panels AC and AD). Note the elevated PCNA staining, compared to the control, in the bronchial epithelium in the presence of 500 and 750 ng/ml Wnt3a and the absence of proliferation at concentrations above 2000 ng/ml.

FIG. 3: Proliferation in fetal lung explants cultured for one day with addition of 0 ng/ml, 30 ng/ml, 125 ng/ml and 250 ng/ml Wnt3A. The number of all cells, including mesenchyme cells, were counted after immunohistochemical staining against phospho histon H3 (ser10).

FIG. 4, Panels A-O: Lung explant culture and subsequent histological analysis four days after the start of the culture in the presence of Wnt3a at a concentration of 0 ng/ml (Panels A-C); 4 ng/ml (Panels D-F); 20 ng/ml (Panels G-I); 100 ng/ml (Panels J-L); and 500 ng/ml (Panels M-O). Note the increasing number of apoptotic cells in the mesenchyme between above 100 ng/ml Wnt3a and the undifferentiated character of the epithelium four days after culture in the presence of 500 ng/ml Wnt3a (Panels M-O).

FIG. 5, Panels A-H: Induction of apoptosis by Wnt3A. The TUNEL assay was performed on histological sections from fetal lung explants, cultured for four days in the presence of Wnt3a at a concentration of 0 ng/ml (Panel A); 0.4 ng/ml (Panel B); 2 ng/ml (Panel C); 10 ng/ml (Panel D); 50 ng/ml (Panel E); 250 ng/ml (Panel F); 500 ng/ml (Panel G) and 1000 ng/ml (Panel H). Note the general increase in apoptotic cells (brown), in the mesenchyme above 100 ng/ml Wnt3A and the massive induction of apoptosis in the mesenchyme in contrast to the unaffected epithelium at 1000 ng/ml, illustrating the difference in sensitivity for Wnt3A induced apoptosis between mesenchyme and epithelium.

FIG. 6, Panels A-H: Effect of Wnt3A on proliferation in mesenchyme and epithelium. The incorporation of BrdU in newly synthesized DNA was analyzed by immunostaining of histological sections from fetal lung explants, cultured for four days in the presence of Wnt3a at a concentration of 0 ng/ml (Panel A); 0.4 ng/ml (Panel B); 2 ng/ml (Panel C); 10 ng/ml (Panel D); 50 ng/ml (Panel E); 250 ng/ml (Panel F); 500 ng/ml (Panel G) and 1000 ng/ml (Panel H).

FIG. 7: The effect of Wnt3A on the final lung differentiation. The lung explants were cultured for eight days in the presence of 0 ng/ml (Panels A and B), 10 ng/ml (Panel C), 50 ng/ml (Panel D), 250 ng/ml (Panel E) and 500 ng/ml Wnt3A (Panel F). After embedding, histological sections were stained with a tubulin-IV antibody to mark ciliated bronchial epithelium. In addition, the sections were counterstained with Haematoxylin and Alcian Blue, which stained the mucus secreted from the bronchial epithelium and the cartilage associated with the tracheal epithelium. Note the abundance of bronchial epithelium, mucus and cartilage in the 500 ng/ml Wnt3A-treated (Panel F) sample. In addition, note the loss of flattened epithelium (alveolar-type-I cells) with increasing dosage of Wnt3A.

FIG. 8: Nuclear translocation of beta-catenin by Wnt3A in fetal lung explants. The translocation of beta-catenin was determined using immunohistological analysis of histological sections from fetal lung explants (E12.5), cultured for two days in the presence of Wnt3a at a concentration of 0 ng/ml (Panel A); 10 ng/ml (Panel B); 50 ng/ml (Panel C); 250 ng/ml (Panel D); 500 ng/ml (Panel E) and 1000 ng/ml (Panel F). Note the strong induction of nuclear beta-catenin in the mesenchyme flanking the epithelium of the growing lung buds at concentrations of 250 ng/ml (Panel D) and 500 ng/ml Wnt3A (Panel E).

FIGS. 9A-9D: Induction of the Wnt-signaling pathway by Wnt3A in human and mouse lung cells. The induction of the Wnt-signaling pathway was measured by performing the TopFlash/FopFlash reporter assay on primary lung fibroblasts isolated from a E12.5 fetal lung (FIG. 9A), the adult human epithelial cell line A549 (FIG. 9B), the fetal human fibroblast cell line HFL-1 (FIG. 9C), and the adult human epithelial cell line calu-3 (FIG. 9D). Note the strong induction of the Wnt-signaling pathway in all three human cell lines, indicating that Wnt3A, like in mouse, is able to induce the Wnt pathway in humans.

FIG. 10: Cluster analysis and the relationship of the expression profiles of 4500 genes significantly changed (p<10⁻⁶) in expression in one of the six fetal lung explant cultures subjected to 0 ng/ml Wnt3A for four hours (0 Wnt/4 hr); 250 ng/ml Wnt3A for four hours (250 Wnt/4 hr); 0 ng/ml Wnt3A for 24 hours (0 Wnt/24 hr); 10 ng/ml Wnt3A for 24 hours (10 Wnt/24 hr); 50 ng Wnt3A for 24 hours (50 Wnt/24 hr); 250 ng Wnt3A for 24 hours (250 Wnt/24 hr). The expression levels of the 4500 genes are placed in six rows in which the expression level of each gene relatively to its mean expression level observed in all six conditions is represented by a colored bar. A green bar corresponds to gene expressed below its mean expression level and a red bar corresponds to genes expressed above its mean expression level. Note the common expression profiles of the fetal lung cultures treated for 24 hours with 10 ng/ml, 50 ng/ml and 250 ng/ml Wnt3A and their closer resemblance with the expression profile of younger untreated lung cultures (0 Wnt/4 hr) than with the expression profile of untreated lung cultures of the same age (0 Wnt/24 hr), indicating that Wnt3A treatment inhibits lung differentiation and development.

### DETAILED DESCRIPTION OF THE PREFERRED EMBODIMENTS

“Lung tissue”, as used herein, comprises at least two or more of the following compartments: the alveolar compartment, the bronchiolar compartment, the bronchial compartment and the tracheal compartment. A “lung cell” is defined herein as an epithelial cell of at least one of these four compartments. A population of lung cells comprises at least two lung cells. In this invention in a preferred embodiment, a population of lung cells together is capable of functioning in the exchange of gasses between the organism's blood compartment and the atmosphere. In the lung, epithelial cells and supporting tissue are present. The supporting tissue comprises connective tissue, nerves and lymph and blood vessels. In this application, connective tissue is described by the term “mesenchyme.”

Omnipotent stem cells are cells that possess the ability to proliferate indefinitely and posses the capacity to differentiate into any possible cell type that is present in the full-grown organism.

Pluripotent stem cell are cells that possess the ability to proliferate indefinitively and possess the capacity to differentiate into a large but limited number of cell types that, in general, belong to a certain tissue, such as the lung, or a certain cell lineage, such as the endoderm, ectoderm or mesoderm.

Stem cells of the invention may be genetically modified and/or they may be obtained by nuclear transfer.

The canonical Wnt-signaling pathway is a signaling cascade in which a collection of proteins controls the proteolytic breakdown of beta-catenin. Some proteins in this pathway are chemically modified by other proteins and/or are capable of inducing other molecules, such as, for instance, sugars and lipids, that often have an influence on the proteolytic breakdown of beta-catenin.

Wnt3a protein is part of a large family of proteins that have a close resemblance to each other in structure and function. Wnt proteins are strongly conserved during evolution in structure and function and are interchangeable between species. The invention, therefore, comprises all Wnt proteins or a functional part, derivative or analogue thereof, that, like Wnt3a, are capable of inducing the canonical Wnt pathway. Other components of the Wnt-signaling pathway or fragments of these components, or compounds mimicking the function of these components, that in the end reduce the proteolytic breakdown of beta-catenin will have the same effect as Wnt3a in kind, not necessarily in amount, and are also within the scope of the present invention.

The terms “Wnt,” “Wnt gene product” or “Wnt polypeptide,” when used herein, encompass native sequence Wnt polypeptides, Wnt polypeptide variants, Wnt polypeptide fragments and chimeric Wnt polypeptides. Optionally, the Wnt polypeptide is not associated with native glycosylation or palmitolyation. “Native glycosylation” refers to the carbohydrate moieties that are covalently attached to Wnt polypeptide when it is produced in the metazoan cell from which it is derived in nature. Native palmitolyation in its turn refers to the covalent attachment of lipid derivatives to a Wnt polypeptide when it is produced in the metazoan cell that is derived in nature (Willert et al., 2003). Accordingly, a human Wnt polypeptide produced in a non-metazoan cell is an example of a Wnt that is “not associated with native glycosylation or palmitolyation.” Sometimes, the Wnt polypeptide is unglycosylated or unpalmitolyated (e.g., as a result of being produced recombinantly in a prokaryote).

A “native sequence” polypeptide is a polypeptide that has the same primary amino acid sequence as a polypeptide (e.g., Wnt polypeptide) derived from nature. Such native sequence polypeptide is, for instance, isolated from nature or is produced by recombinant or synthetic means. A native sequence polypeptide has the amino acid sequence of naturally occurring human polypeptide, murine polypeptide, or polypeptide from any other mammalian species.

The term “native sequence Wnt polypeptide” includes Wnt polypeptides from any animal species (e.g., human, murine, rabbit, cat, cow, sheep, chicken, porcine, equine, etc.) as occurring in nature. The term “native sequence Wnt protein” includes the native proteins with or without the initiating N-terminal methionine (Met) and with or without native signal sequence. The native sequence human and murine Wnt polypeptides known in the art are from about 348 to about 389 amino acids long in their unprocessed form reflecting variability (particularly at the poorly conserved amino-terminus and several internal sites), contain 21 conserved cysteines, and have the features of a secreted protein (see, e.g., Wnt polypeptides as in Gavin et al., supra; Lee et al., supra; Christiansen et al., supra; PCT/US94/14708 (WO 95/17416)). The molecular weight of a Wnt polypeptide is about 38 to 42 kD in a monomeric form.

A “functional part, derivative or analogue” of a Wnt polypeptide means a biologically active polypeptide as defined below having less than 100% sequence identity with a native sequence Wnt polypeptide and having the same activity in kind, not in amount. Such functional parts, derivatives or analogues include polypeptides, wherein one or more amino acid residues are added at the N- or C-terminus of, or within, the native sequence; polypeptides, wherein from about one to forty amino acid residues are deleted and optionally substituted by one or more amino acid residues; and derivatives of the above polypeptides, wherein an amino acid residue has been covalently modified so that the resulting product has a non-naturally occurring amino acid. Preferably, a biologically active Wnt variant has an amino acid sequence having at least about 90% amino acid sequence identity with a native sequence Wnt polypeptide, preferably, at least about 95%, more preferably, at least about 99%. A “functional part” means having an effector function that is directly or indirectly caused or performed by native sequence Wnt polypeptide, such as Wnt3a. Effector functions of native sequence Wnt polypeptides preferably include inhibition of differentiation and/or enhancement of proliferation and/or induction of apoptosis.

“Functional part or derivatives” include, but are not limited to, fragments of a native Wnt polypeptide sequence and derivatives of a native sequence Wnt polypeptide and its fragments, provided that they have a biological activity in common with a corresponding native sequence Wnt polypeptide. The term “derivative” encompasses both amino acid sequence variants of Wnt polypeptide and covalent modifications thereof.

“Isolated” Wnt polypeptide has been purified from a Wnt source or has, for instance, been prepared by recombinant or synthetic methods and is sufficiently free of other peptides or proteins (1) to show homology for at least 15 and preferably 20 amino acid residues of the N-terminal or of an internal amino acid Wnt polypeptide sequence, or (2) to homogeneity by SDS-PAGE under non-reducing or reducing conditions using Coomassie blue or, preferably, silver stain.

The term “antibody” is used for binding molecules in the broadest sense and, amongst other things, covers monoclonal antibodies, antibody compositions with poly-epitope specificity, bispecific antibodies, diabodies, and single-chain molecules, as well as antibody fragments (e.g., Fab, F(ab′).sub.2, and Fv), so long as they exhibit the desired biological activity.

The term “monoclonal antibody,” as used herein, refers to an antibody obtained from a population of substantially homogeneous antibodies, i.e., the individual antibodies constituting the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the use of monoclonal antibodies synthesized by a hybridoma culture is advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention are in one embodiment made by the hybridoma method first described by Kohler et al., Nature 256:495 (1975), and are in another embodiment made by recombinant DNA methods (see, e.g., U.S. Pat. No. 4,816,567 (Cabilly et al.)). In yet another embodiment, the “monoclonal antibodies” are isolated from phage antibody libraries using the techniques in Clackson et al., Nature 352:624-628 (1991), and Marks et al., J. Mol. Biol. 222:581-597 (1991), for example.

The monoclonal antibodies as provided herein also comprise “chimeric” antibodies (immunoglobulins) in which a portion of the heavy and/or light chain is identical with or homologous to corresponding sequences in antibodies derived from a particular species or identical with or homologous to corresponding sequences in antibodies derived from another species or belonging to another antibody class or subclass, as well as fragments of such antibodies, so long as they exhibit desired biological activity (Cabilly et al., supra; Morrison et al., Proc. Natl. Acad. Sci. USA, 81:6851-6855 (1984)).

The phrase “enhancement of proliferation of a cell” encompasses the step of increasing the extent of growth and/or reproduction of the cell, relative to an untreated cell either in vitro or in vivo. An increase in cell proliferation in cell culture is, for instance, detected by counting the number of cells before and after exposure to a molecule of interest. The extent of proliferation is alternatively quantified via microscopic examination of the degree of confluency. Cell proliferation is also quantified using a thymidine or BrdU incorporation assay.

By “controlling differentiation of a cell” is meant the act of increasing the extent of the acquisition or possession of one or more characteristics or functions that differ from that of the original cell (i.e., cell specialization). This is, for instance, detected by screening for a change in the phenotype of the cell (e.g., identifying morphological changes in the cell and/or surface markers on the cell).

A “lung stem cell or lung progenitor cell” or “primitive lung cell” is a cell that is able to differentiate to form a more committed or mature lung cell type.

“Mammal” refers to a human or non-human mammal, including, a domestic and farm animal, and a zoo, a sports, or a pet animal, such as a dog, a horse, a cat, a cow, etc. Preferably, the mammal is human.

The invention is further described with the aid of the following illustrative Examples.

### EXAMPLES

### Example 1

### Generation of New Lung Tissue Ex Vivo

Preservation and Potential Growth of Alveolar Tissue in a Murine Lung by Culture Treatment with Recombinant Wnt3a Polypeptide

To provide proof of evidence, an in vitro explant lung culture (obtained from a mouse) is used. Generation of new alveolar tissue in patients is stimulated by activation or re-activation of alveolar bud formation. Growth and branching of alveolar buds, followed by transformation into alveolar ducts, sacs and pouches, results in the establishment of pulmonary acini.

This is a general differentiation principle in both the fetal and the adult mammal.

As a proof of evidence, it is, therefore, shown that treatment with selected molecules involved in a Wnt pathway inhibits the differentiation process of alveolar differentiation. Inhibition of this differentiation process is achieved by applying a purified Wnt3a polypeptide to an in vitro lung explant culture of 13-day-old (E13) mouse embryos.

### Material and Methods

### In Vitro Lung Explant Cultures

The (fetal) murine lung explant culture was generated according to our standard protocol. Briefly, complete lungs or individually dissected lung lobes were isolated from fetal mice and cultured for one to five days in a hanging drop culture system. The Wnt3a polypeptide was administered to the culture medium in different concentrations, ranging from 0 to 4000 ng/ml. The effect of this treatment was monitored by stereomicroscopy. The lung explant cultures were subsequently fixed for 30 minutes in 10% paraformaldehyde (PFA), 30 minutes in 2% PFA and 30 minutes in 4% PFA. After fixation, the lung explants were dehydrated through an increasing series of 2-isopropanol and subsequently embedded in paraffin. For the histological analysis, 4 μm thick sections were cut and stained with Hematoxilin and Eosin using standard techniques.

For the immunohistochemical analysis, comparable sections were used and treated with an anti-PCNA monoclonal antibody followed by a goat anti-mouse horseradish peroxidase conjugate. Detection of the PCNA epitopes was carried out by treating the sections with di-aminobenzidine (DAB) for 15 minutes.

### TopFlash/FopFlash Reporter System

Primary mouse fibroblasts isolated from E12.5 lung explants, the human epithelial lung carcinoma cell line A549 cells (ATCC CCL-185), the human epithelial lung adenocarcinoma cell line Calu-3 (ATCC_HTB-55) and the normal human fetal lung fibroblast cell line HFL-1 cells (ATCC_{—}153) were cultured in 96-well plates and transfected with the TopFlash and FopFlash reporter constructs using lipofectamine 2000 according to the manufacturer's instructions. As an internal control for transfection efficiency, a constitutive renilla luciferase reporter (Promega) was cotransfected. The cells were transfected with the above-mentioned reporter constructs and simultaneously treated with different concentrations of Wnt3A for 20 hours, after which the cells were lysed and luciferase expression was measured using a Berthold luminometer for two seconds (V. Korinek et al., Mol. Cell. Biol. 18:1248-1256, 1998). The Lipofectamine 2000 transfection reagent was purchased from Invitrogen Life Technologies. The wild-type beta-catenin/TCF reporter plasmid (TOPFLASH) and mutated reporter plasmid (FOPFLASH) were obtained from Upstate Biotechnology (NY, USA).

### Quantification of Cell Proliferation Using Phospho Histon H3 Immunohistochemistry

The number of cells undergoing mitosis was measured using immunohistochemical detection of phosphorylated histon H3, which is present only on condensed chromosomes during mitosis. Per condition, at least four different lung explants were treated and in total, 20 sections per condition were counted for phospho histon H3-positive cells by two different investigators. The primary antibody against phopho histon H3 (Ser10, clone 6G3) was purchased from Cell Signaling Technology and used according to the recommendations of the supplier.

The induction of apoptosis was detected using the “In Situ Cell Death Detection Kit,” of Roche (Cat. No. 11 684 817 001) according to the manufacturer's instructions.

As a source for Wnt3a, recombinant mouse Wnt3a from R&D Systems (catalog nr. 1324-WN, R&D Systems, Inc. Minneapolis USA) was used. As culture medium, DMEM/HAM's F12 (1:1) from Biochrom AG, catalog nr. FG 4815, was used.

The antibody against PCNA was purchased from Zymed Laboratories Inc. (cat nr. 18-0110). The secondary conjugate used was from Jackson Laboratories, Inc. (catalog nr. 115-056-062).

The primary antibodies used for the immunohistochemical detection was for beta-catenin; anti beta-catenin IgG, mAb, clone 14 from BD-Transduction Laboratories, for tubulin-beta mAb clone TBN05 (Tub2.1) from Neomarkers, for BrdU; anti-Bromodeoxyuridine (BrdU), mAb clone ZBU30 from Zymed Laboratories Inc.

The Effect of 500 ng/ml to 4000 ng/ml Wnt3A on the Growth of Fetal Lung Explants Results:

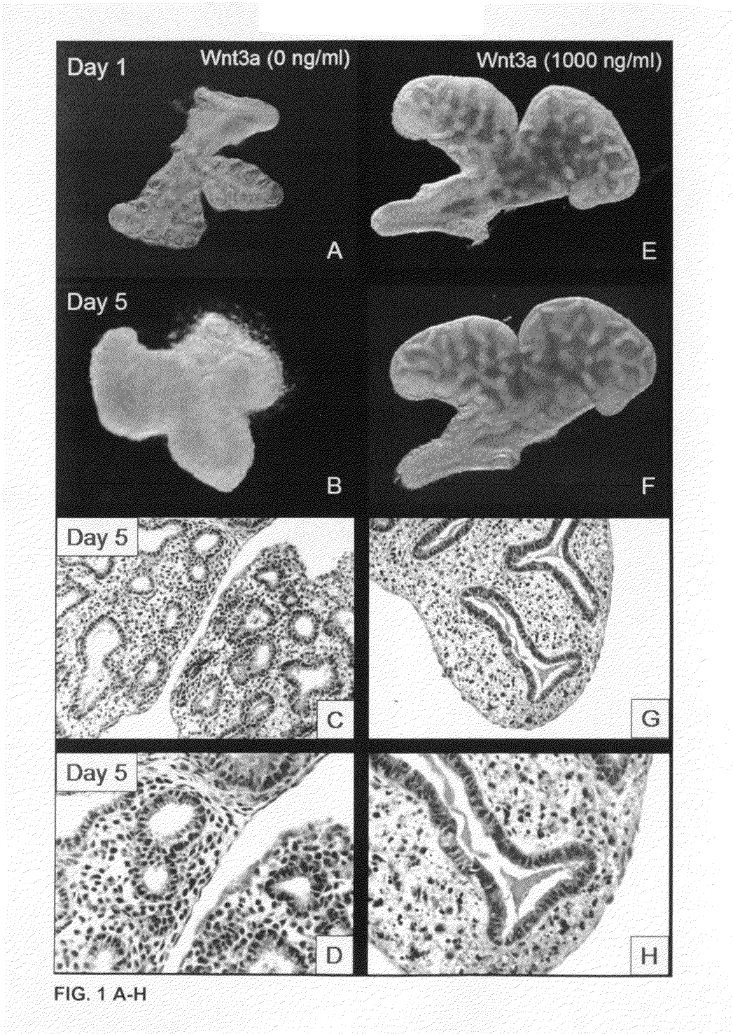

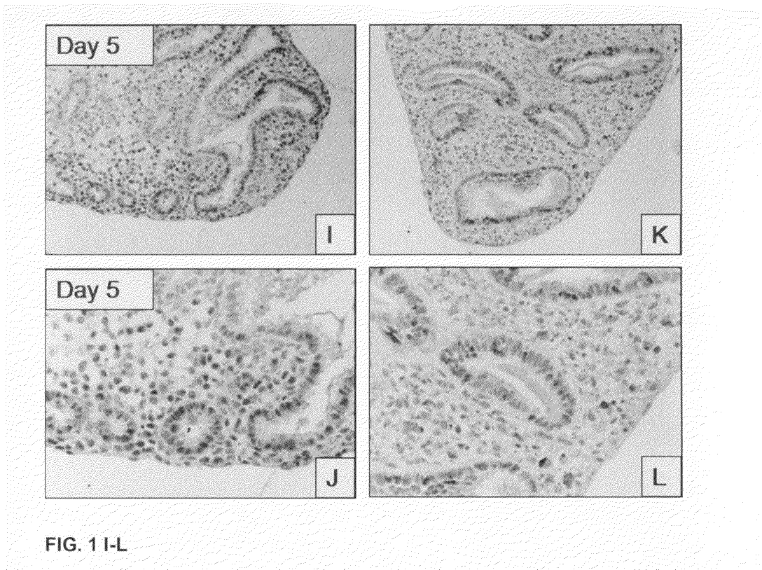

Treatment of the lung cultures of embryonic day 13 (E13) lung explants with 1000 ng/ml Wnt3a polypeptide resulted in a total block of lung differentiation after 16 hours (FIG. 1, Panel E). Even after five days culture under these conditions, no significant differentiation and alveolar development could be observed by stereomicroscopy (FIG. 1, Panel F).

Histological analysis of these five-day-old cultures revealed that the differentiation of the primordial lung epithelium was arrested at a stage comparable to that of the start of the culture (E13). Furthermore, most of the mesenchymal cells surrounding the epithelium displayed picnotic nuclei, indicating that these cells had undergone apoptosis. This was in sharp contrast with the epithelium that contained no apoptotic cells (FIG. 1, Panel H).

To analyze the viability of the epithelium and the surrounding mesenchyme, the sections were stained for the proliferation marker “Proliferating Cell Nuclear Antigen” (PCNA, FIG. 1, Panels I-J). This immunohistochemical staining showed that the epithelium was still proliferating whereas most of the mesenchymal cells were inactive or dead (FIG. 1, Panels K and L). This is in contrast to the untreated lung explant cultures where both the epithelium and the mesenchyme were strongly proliferating (FIG. 1, Panels I and J).

The above results indicate that the Wnt3a polypeptide has a strong inhibitory effect on the development and differentiation of the fetal lung.

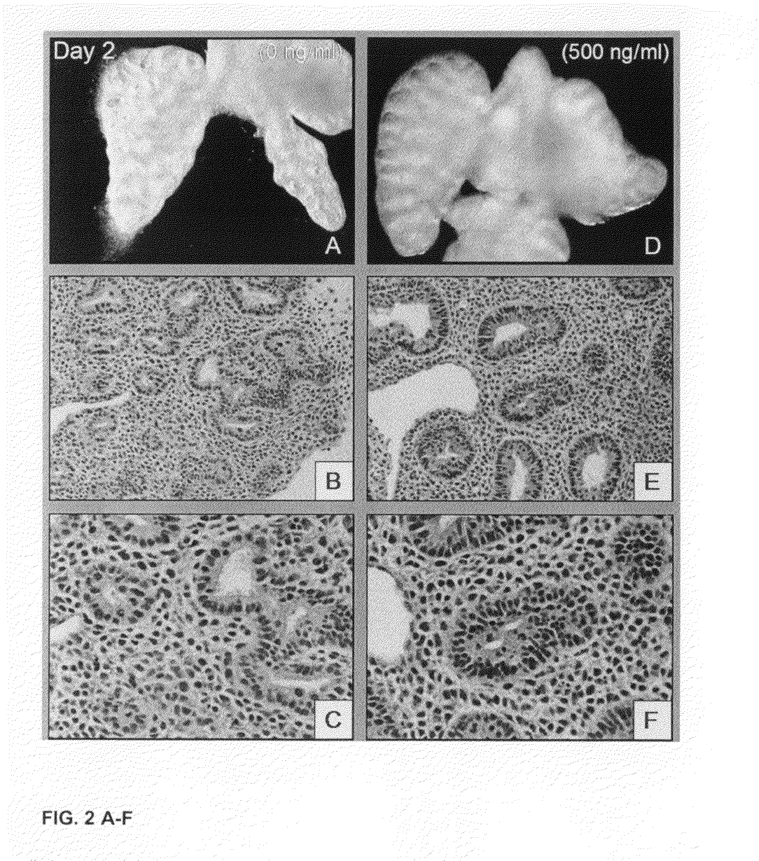

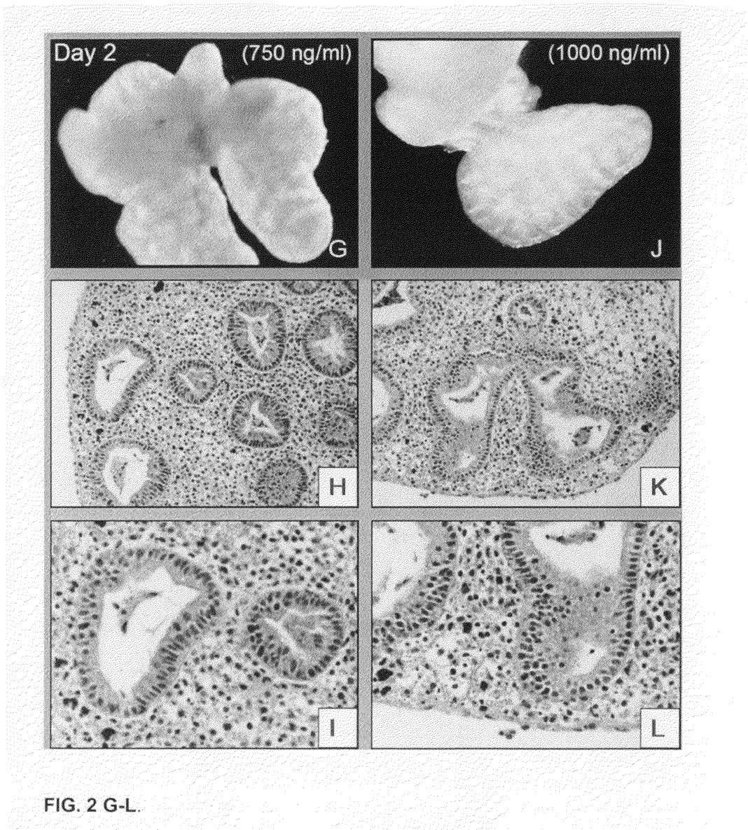

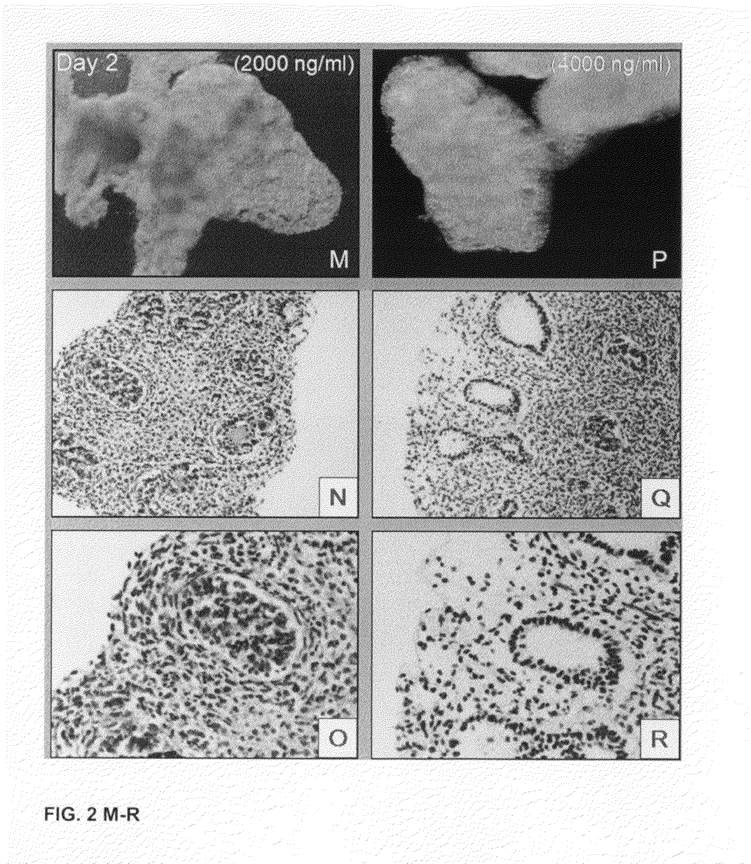

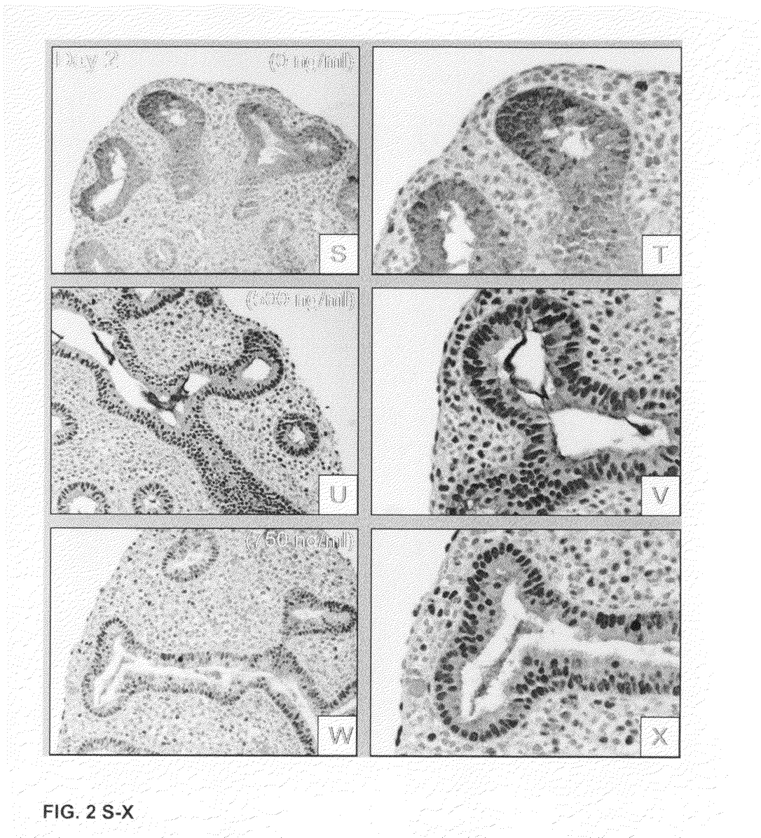

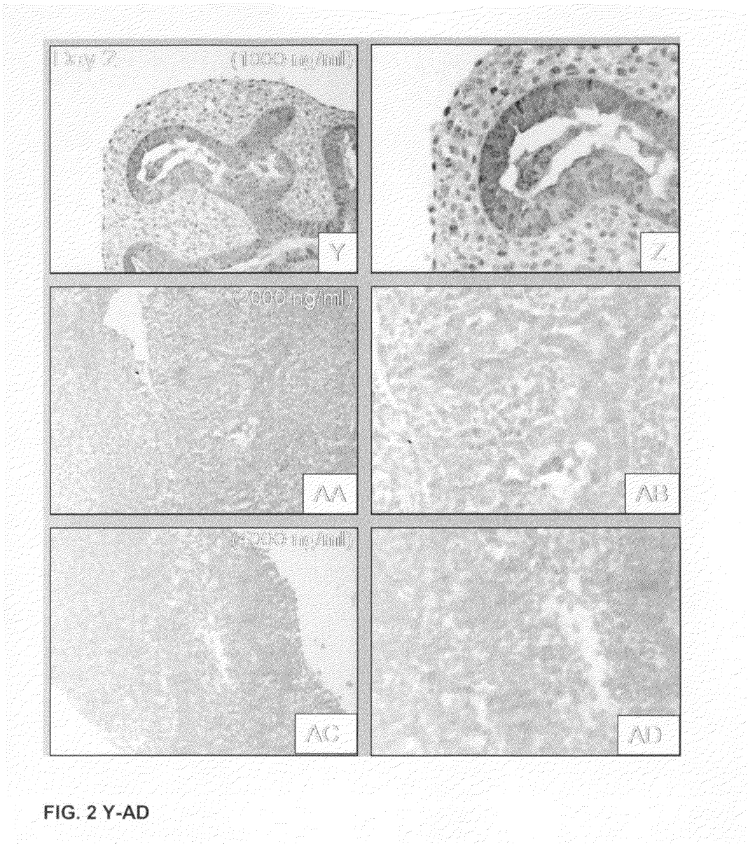

In order to determine the concentration range in which the Wnt3a polypeptide is effective, total lung explants were cultured in the presence of Wnt3a in a range of 0 ng/ml, 500 ng/ml, 750 ng/ml, 1000 ng/ml, 2000 ng/ml and 4000 ng/ml (FIG. 2, Panels A-R).

Stereomicroscopic analysis showed already after one day a deleterious effect on lung development with the Wnt3a concentrations, 2000 ng/ml and 4000 ng/ml (FIG. 2, Panels M and P).

After two days of culture, these lung explants were fixed and embedded in paraffin for immunohistological analysis.

The histological analysis of H/E stained sections showed that the mesenchymal cell population undergoes a rapid induction of apoptosis within the range 1000 to 4000 ng/ml Wnt3a. Apoptosis is also induced in the epithelium at a concentration of 2000 to 4000 ng/ml but is not detectable at a concentration of 1000 ng/ml. The mesenchyme, however, at a concentration higher than 750 ng/ml, becomes clearly apoptotic, indicating that the induction threshold for apoptosis is different for the epithelium and the mesenchyme. At the concentration of 750 ng/ml, apoptosis in the mesenchyme is clearly detectable but not yet abundant, whereas the epithelium showed no sign of apoptosis whatsoever FIG. 2, Panels H and I).

Immunohistochemical staining of PCNA in addition showed a Wnt3a-dependent proliferation upon the lung explant cultures. Without addition of Wnt3a, the periphery of the lung containing the growing lung buds show a stronger staining for PCNA than the more centrally located bronchial epithelium, indicating that the periphery of the lung is stronger proliferating than the central part (FIG. 2, Panels S and T). Addition of 500 ng/ml Wnt3a to the medium, however, shows a marked overall increase in the intensity and frequency of PCNA staining in the centrally located bronchial epithelium, indicating that proliferation is enhanced by Wnt3a at a dosage of 500 ng/ml Wnt3a (FIG. 2, Panels U and V). A further increase of the Wnt3a concentration to 750 ng/ml (FIG. 2, Panels W and X) does not lead to a further increase of the intensity and frequency of the PCNA staining, but instead, is comparable to the control situation (FIG. 2, Panels S and T). Addition of 1000 ng/ml Wnt3a reduces the PCNA staining below the control situation (FIG. 2, Panels Y and Z), whereas at 2000 and 4000 ng/ml Wnt3a (FIG. 2, Panels AA and AD), PCNA staining was completely negative indicating that proliferation was completely blocked, probably due to the apoptosis induced in the mesenchyme and epithelium (FIG. 2).

To further determine the short-term effect of different Wnt3A concentrations upon the induction of proliferation, lung explants (E12.5) were cultured for one day with 0, 30, 125 and 500 ng/ml Wnt3a.

The lung explants were processed for immunohistochemical analysis and stained for Histon H3 phosphorylation, which can be used as a quantifiable cell proliferation marker as it is present in a fixed amount on condensed chromosomes only during active cell division (material and methods). The results of this quantification show that Wnt3A has a significant stimulatory effect upon proliferation. With more than a 20% increase overall, proliferation was observed in the 30, 125 and 500 ng/ml-treated Wnt3A samples. However, most of the increase in proliferation could be accounted to the mesenchyme, where an increase in 40 to 60% of cells undergoing apoptosis was observed. Little difference was found between the different Wnt3A concentrations, indicating that the maximum stimulatory effect of Wnt3A was already reached with 30 ng/ml Wnt3a (FIG. 3).

The Effect of 4 ng/ml to 500 ng/ml Wnt3A on Fetal Lung Growth Over Four Days

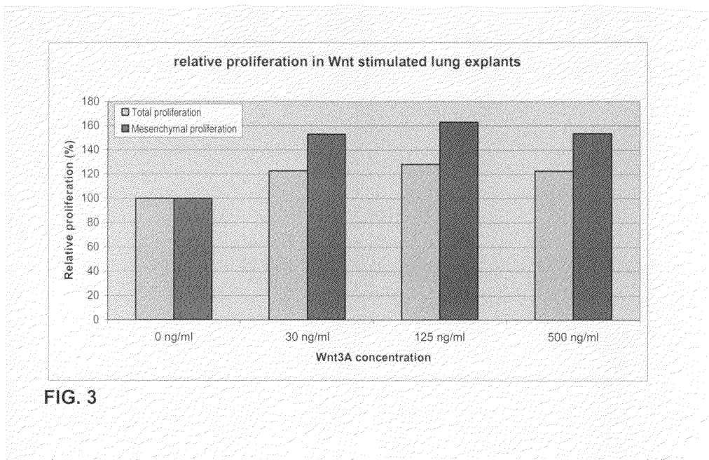

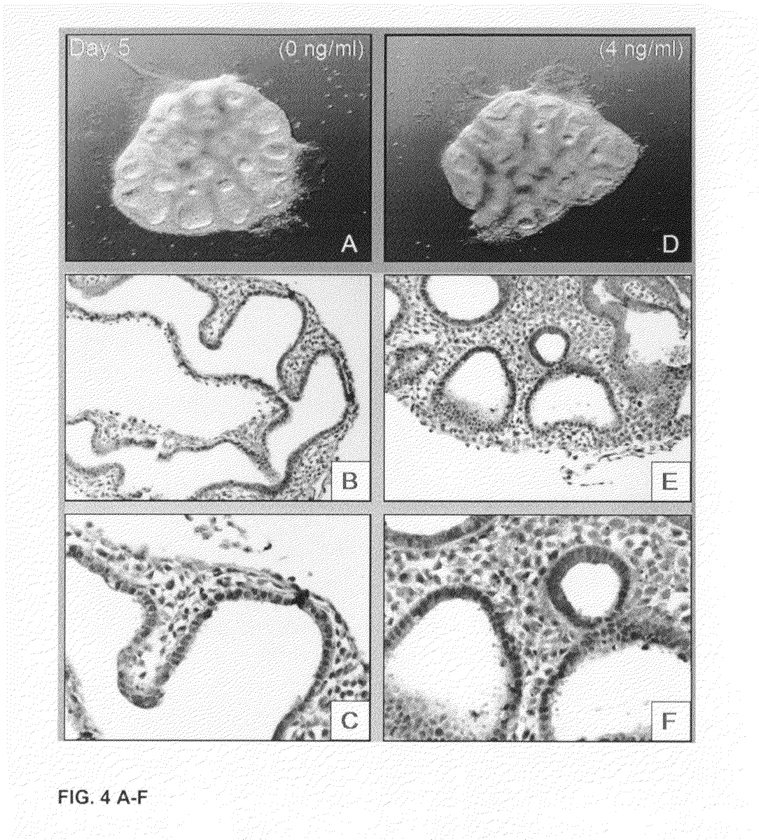

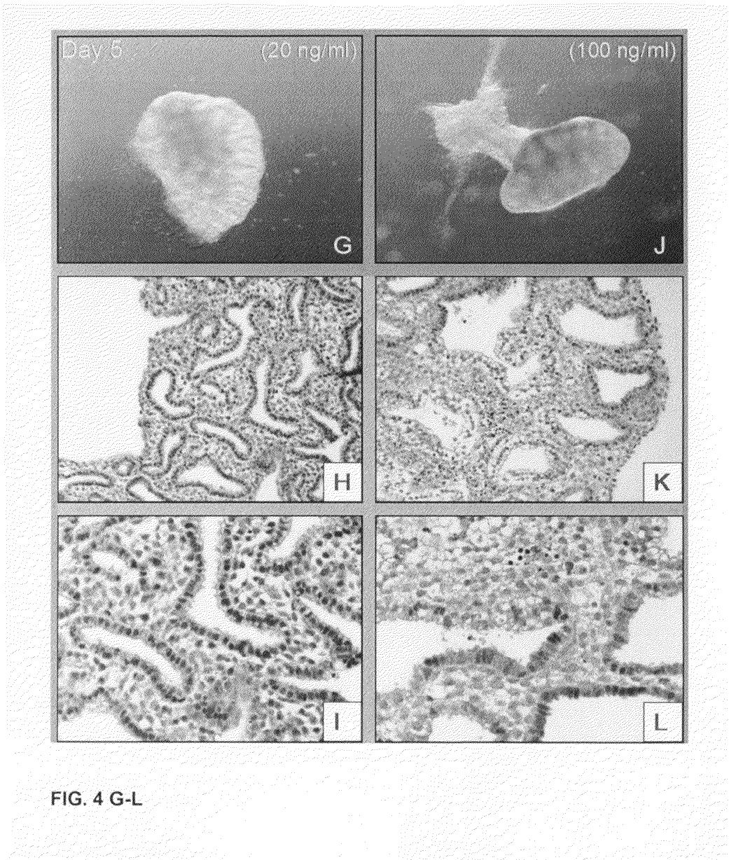

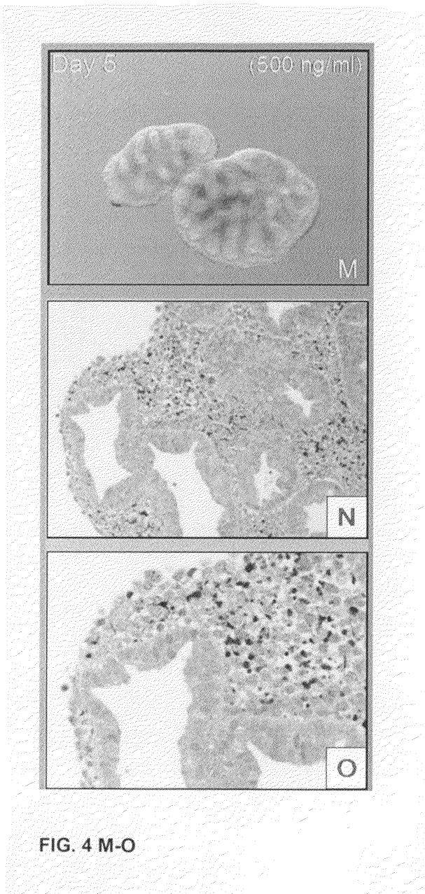

To more exactly determine the concentration of Wnt3a that is effective upon lung development, differentiation and apoptosis, fetal lung explants were cultured for a longer period (four days) in the presence of lower concentrations of Wnt3a, i.e., 0 ng/ml, 4 ng/ml, 20 ng/ml, 100 ng/ml and 500 ng/ml (FIG. 4, Panels A-O).

None of these concentrations was effective in completely blocking the differentiation of fetal lung development, although differentiation at a concentration of 500 ng/ml (FIG. 4, Panels N and O) was delayed as was displayed by the primordial morphology of the epithelium compared to the control situation (FIG. 4, Panels B and C). Between 100 and 500 ng/ml, the mesenchyme showed an increasing level of apoptosis, whereas in the epithelium, no sign of apoptosis could be detected (FIG. 4, Panels K, L, N and O).

Between 4 and 20 ng/ml, no clear effects were visible at the level of apoptosis and differentiation compared to the control situation (FIG. 4, Panels B, C, E, F, H, and I).

Analysis of Apoptosis by the TUNEL Assay and Active Proliferation by BrdU Incorporation on Lung Explants Treated with 4 ng/ml to 1000 ng/ml Wnt3A During Four Days

The above-described experiments show that the visible biological effect of Wnt3A upon proliferation, apoptosis, and differentiation lies between a concentration range of 1 and 2000 ng/ml. A concentration of 2000 ng/ml is not preferred since it affects the viability of all cells, and thus lacks specificity. A concentration below 1 ng/ml does not show clear morphological changes, although a concentration of 30 ng/ml markedly increases proliferation after one day. Drastic effects were observed at the level of apoptosis, proliferation and differentiation. To further analyze the effect of Wnt3a upon apoptosis and proliferation, lung explants were cultured for four days in the presence of 0, 4, 20, 100, 250, 500, 750 and 1000 ng/ml. In order to identify whether after Wnt3A treatment cells are viable and active, proliferating BrdU was added to the culture medium, which is incorporated in newly synthesized DNA of active proliferating cells, two hours before the fixation of the lung explants. Histological examination at day 4 showed, as before, that the process of differentiation was strongly retarded and revealed many picnotic cells at Wnt3a concentrations above 100 ng/ml. To better investigate this effect, histological sections were analyzed with the TUNEL assay, which detects fragmented DNA present in apoptotic cells (material and methods). A low background of TUNEL-positive cells was detected in the sham-treated lung explants, however, a dramatic increase in TUNEL-positive cells was observed at concentrations above 100 ng/ml Wnt3a, which is proportional to the number of picnotic cells found in these samples (FIG. 5). At 1000 ng/ml, almost 100% of the mesenchymal cells were picnotic and were also TUNEL positive, indicating that they had undergone apoptosis (FIG. 4, Panel H). In contrast, almost none of the epithelial cells were TUNEL positive, or contained picnotic nuclei, indicating that these cells are much less sensitive for apoptosis compared to the mesenchymal cells (FIG. 5).

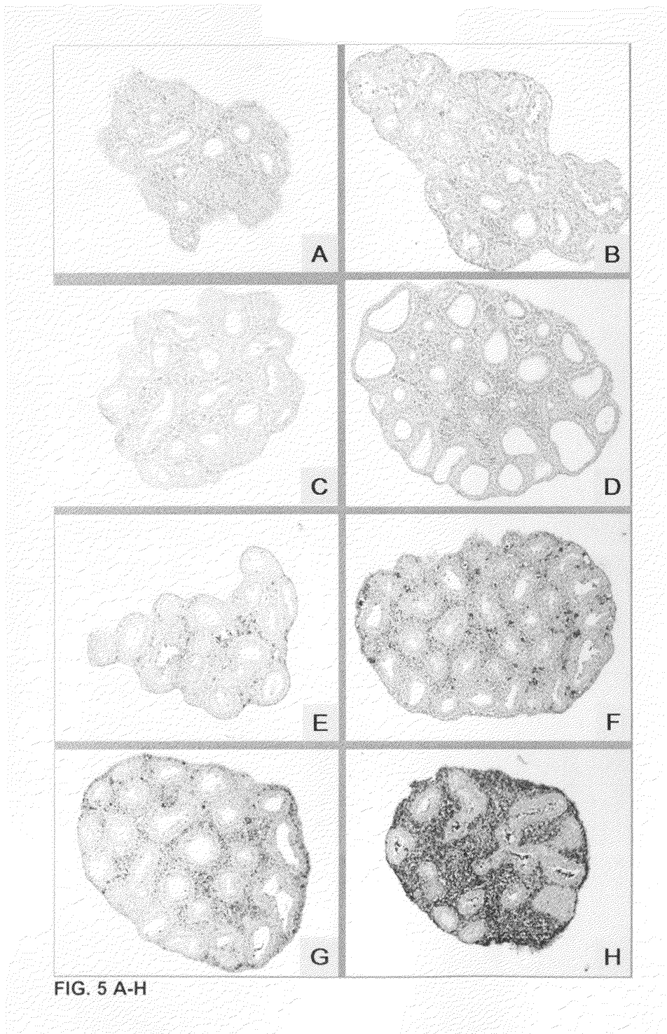

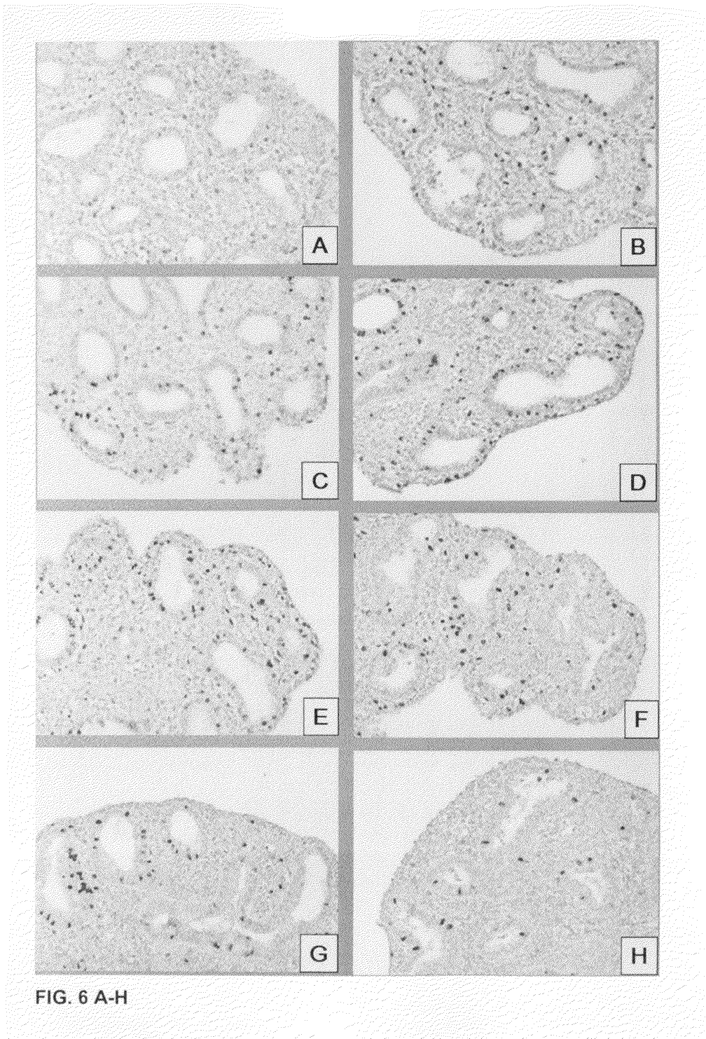

In order to identify whether the epithelial cells were still actively proliferating after four days of Wnt3a treatment, the histological sections were immunostained for incorporated BrdU (FIG. 6). At concentrations of 500 ng/ml and lower, both the mesenchyme and epithelium contained many BrdU-positive, thus active, proliferating cells. At 1000 ng/ml, almost all BrdU-positive cells were present in the epithelium and only a very few were present in the mesenchyme, indicating that despite the massive induction of apoptosis in the mesenchyme, the epithelium is viable and proliferating. At 750 ng/ml Wnt3a, an intermediate effect was observed between the 500 and 1000 ng/ml Wt3A-treated lung explants (FIG. 6).

Long-Term Treatment of Fetal Lung Explant Cultures with Wnt3a Induces a Dosage-Dependent Shift of Distal (Alveolar) Lung Differentiation to Proximal (Bronchial/Tracheal) Lung Differentiation

To further determine the effect of Wnt3A upon the development and differentiation of the lung over time, fetal lung explants of isolated E12.5 were cultured with 0, 10, 50, 250, and 500 ng/ml Wnt3A for eight days. Two hours before the fixation of the lung explants, BrdU was added to the culture medium, which is incorporated in newly synthesized DNA of active proliferating cells. After the different time points, the lung explants were processed for histological analysis and immuno staining of marker genes.

Eight-Day Culture without Wnt3A

The histological analysis showed that the long-term lung development in the hanging drop culture system, without Wnt3A addition to the medium, is comparable to in vivo lung development. The lung explants without Wnt3A could be cultured for eight days without the sign of necrosis or apoptosis. Contraction of the bronchial epithelium is visible after one to two days of culture, indicating that smooth muscle cells develop along the conducting airways. This was confirmed with immunohistochemical staining for smooth-muscle actin (data not shown). Several differentiated cell types, such as cubical type-II cells, secretory Clara cells, cartilage and the alveolar-type-I cells could be clearly distinguished after eight days (FIG. 7, Panel A). This is not much delayed from the in vivo situation where alveolar-type-I cells differentiate between E17.5 and E19.5, thus corresponding to days five and seven of the in vitro culture.

Eight-Day Culture with 10 to 250 ng/ml Wnt3A

Treatment of the E12.5 day lung explants showed a clear change in morphology, which became more and more apparent over time, indicating that a Wnt3A concentration as little as 10 ng/ml has profound effects on lung development. Histological analysis of these explants after eight days showed a decreasing alveolarization of the distal epithelium. The normal flattening of the primordial epithelium to the type-I cell that occurs during normal distal lung differentiation is inhibited with increasing Wnt3A concentration and, at 250 ng/ml, it is almost completely suppressed (FIG. 7, Panels B-E). Distal lung development is thus inhibited by Wnt3A treatment in a concentration range of at least 10 ng/ml to 250 ng/ml Wnt3A.

Eight-Day Culture with 500 ng/ml Wnt3A

Addition of Wnt3A has a striking effect on the final differentiation of E12.5 lung explants. Previous cultures had already shown the short-term inhibitory effect upon branching of 500 ng/ml Wnt3A after one day. After eight days treatment with 500 ng/ml Wnt3A, branching was still severely suppressed and in morphology, resembled the original isolated E12.5 lung explants. However, histological analysis and immunostaining with an anti-tubulin IV antibody that marks ciliated epithelium, showed that these explants consist completely of proximal structures comprising bronchial epithelium composed of ciliated cells and secretory cells, cartilage and mesenchyme. No type-I or type-II cells could be observed indicating that the differentiation of distal epithelium comprising the alveolar and bronchiolar compartments was completely inhibited by the treatment with 500 ng/ml Wnt3A (FIG. 7, Panel F).

The administration of concentrations higher than 250 ng/ml Wnt3A thus restricts lung differentiation to cell types found in the upper airways and is thus suitable for selectively growing proximal lung cell types from primordial lung tissue, an application which is very useful in generating tissue to treat diseases or injuries of the upper airways.

### Wnt3a Induces the Accumulation of Nuclear Beta-Catenin in Lung Mesenchymal Cells

In order to determine whether Wnt3A actually elevates the Wnt-signaling pathway in these explants, histological sections were stained for beta-catenin, the intracellular factor, which, together with members of the high mobility group (HMG) of transcription factors such as TCF1 and LEF, transducer the Wnt signal to the nucleus.

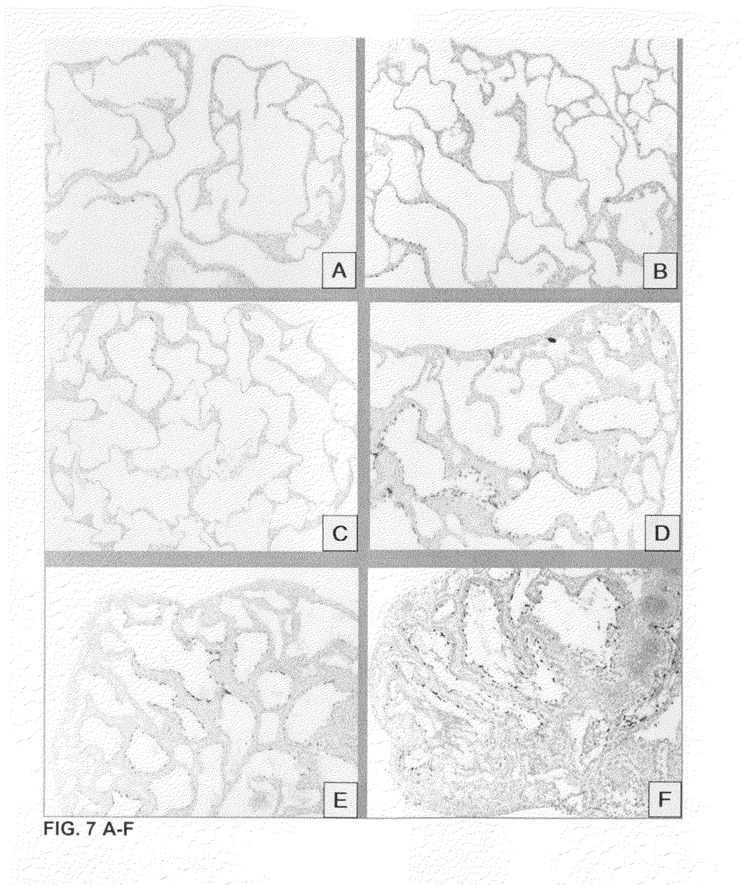

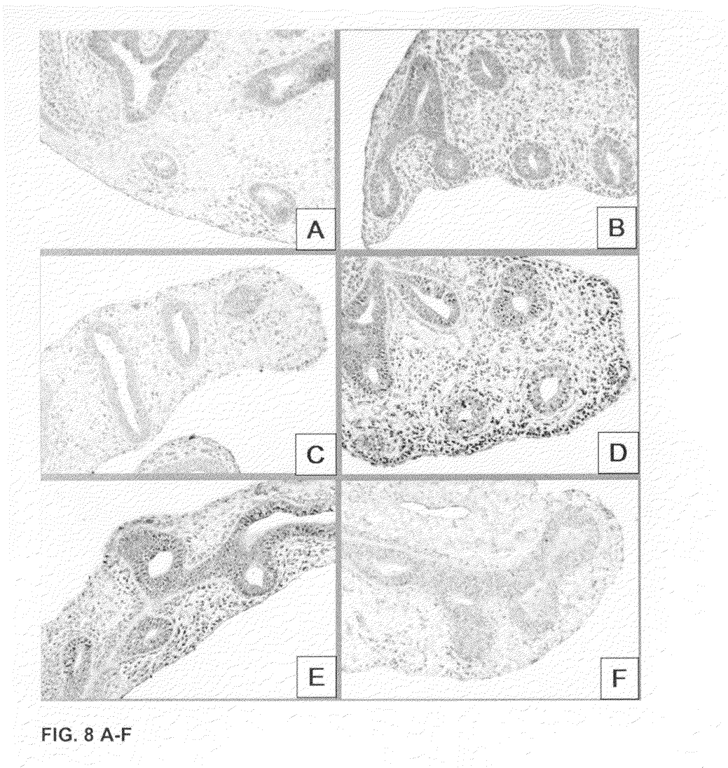

The results showed that there is a Wnt3A dosage-dependent increase in nuclear beta-catenin within the first two days, which was especially prominent in the mesenchymal cells surrounding the growing and branching lung buds in the distal lung compartment. This effect was maximal in the 250 and 500 ng/ml Wnt3A-treated lungs (FIG. 8, Panels D and E), and had disappeared in the four- and eight-day cultures (data not shown).

### Wnt3a Induces a Dosage-Dependent Activation of Wnt-Reporter Expression in Fetal Lung Tissue

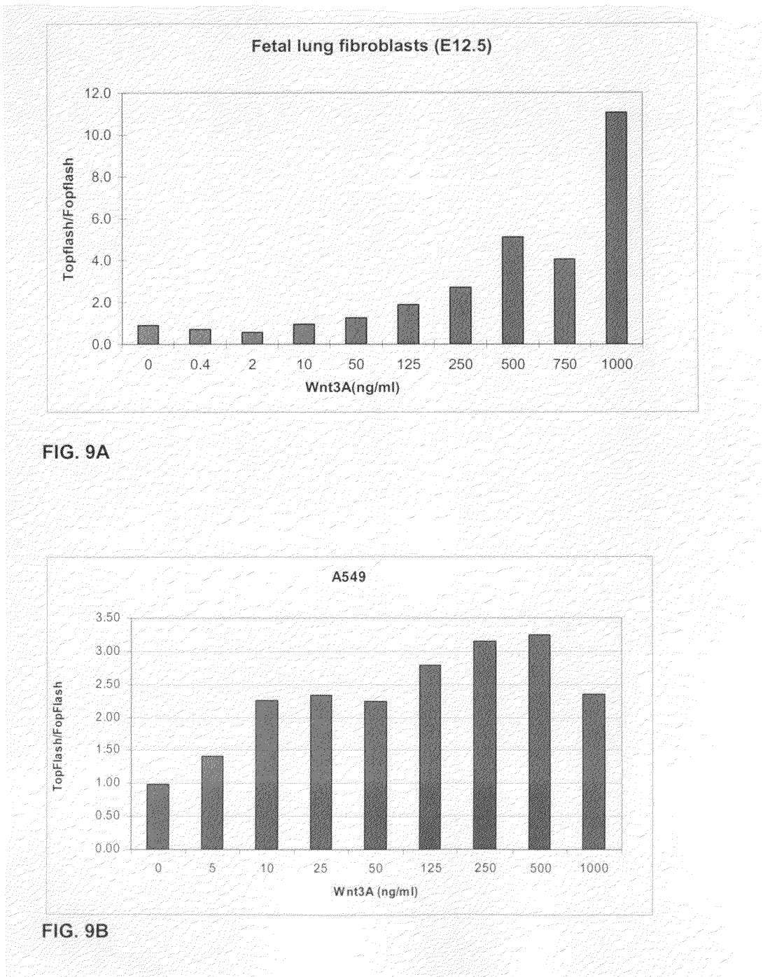

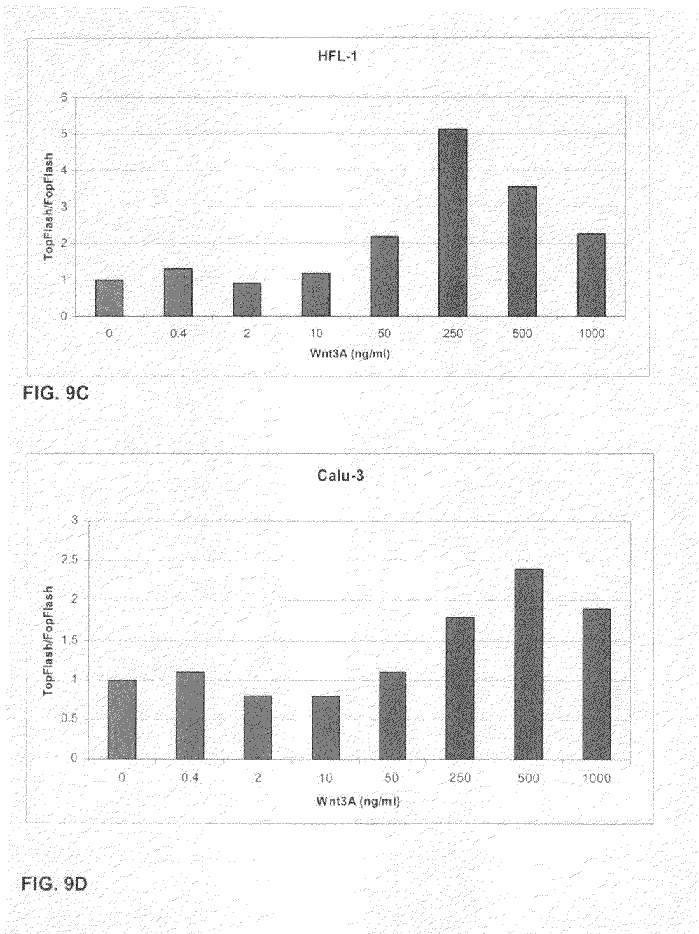

The above results indicate that fetal lung tissue is capable of transducing the Wnt signal by addition of Wnt3A to the culture. To further prove that Wnt3A is capable of inducing the wnt-signaling pathway in lung tissue, we conducted the TopFlash/FopFlash reporter assay on dissociated lung explants isolated from E12.5 embryos. The dissociated lungs were co-transfected with the TopFlash reporter construct that is activated by nuclear beta-catenin transcription complexes. This was compared to the activation of a FopFlash reporter construct that is mutated in the beta-catenin/TCF-binding sites and measures only the background expression from the minimal promoter (V. Korinek et al., Mol. Cell. Biol. 18:1248-1256, 1998). The ratio TopFlash expression versus FopFlash expression is a measure for the induction of the Wnt-signaling pathway by Wnt3A. The transfected cells were treated with different concentrations of Wnt3A ranging between 0.4 and 500 ng/ml Wnt. A clear Wnt3A dosage-dependent response was observed, indicating that the fetal lung cells have all the necessary components to receive and transduce the wnt signal-transduction pathway (FIG. 9A).

In order to analyze if Wnt3A is able to activate the Wnt signal-transduction pathway in humans, we stimulated three different cell lines from human origin with Wnt3A and measured the response with the TopFlash/FopFlash reporter assay. The three human lung-derived cell lines analyzed were the lung epithelial carcinoma cell line A549 (FIG. 9B), the normal lung fibroblast line HFL-1 (FIG. 9C), and the epithelial adenocarcinoma cell line calu-3 (FIG. 9D). The results revealed that all three cell lines respond to Wnt3A in a dosage-dependent manner, indicating that human and mouse lung cells are able to receive and transduce the Wnt signal upon stimulation with Wnt3A.

Expression Profiling Demonstrates that Wnt3A Inhibits the Differentiation of Fetal Lung Explants

To further investigate the effect of Wnt3A inhibition on lung differentiation, we analyzed the expression profiles of Wnt3A-treated lung explants with microarray analysis using the Affymetrix chip set MOE430AB, containing the complete mouse transcriptome. The lung explants, per condition, ten complete fetal lungs isolated from E12.5 embryos, were treated for four hours with 0 and 250 ng/ml Wnt3A (Wnt0/4 hr and Wnt250/4 hr, respectively), and for 24 hours with 0, 10, 50 and 250 ng/ml Wnt3A (Wnt10/24 hr, Wnt10/24 hr, Wnt50/24 hr, Wnt250/24 hr, respectively). The 60 lung explants were isolated together from six pregnant Swiss mice and randomly distributed over the six different conditions.

After the indicated time period, total RNA was isolated, which was processed for the synthesis of a probe for hybridization onto the MOE-430AB chip sets. The results show a strong effect of Wnt3A upon gene expression in the lung explants. A rapid response was observed when the lungs were treated for four hours with 250 ng/ml Wnt3A. A large number of genes were differentially expressed more than two-fold between the Wnt250/4 hr group and Wnt0/4 hr group, i.e., 446 up-regulated genes and 1165 down-regulated genes. In accordance with the expectations, many known Wnt-responsive genes were found in the up-regulated gene pool, again showing that the Wnt pathway was activated in these lung explants.

In the 24-hour-treated explants, a less pronounced effect was observed. In the ng/ml wnt3a-treated group, 209 genes were up-regulated and 223 genes were down-regulated more than two-fold. In the 50 ng/ml-treated group, 95 and 92 genes were up-regulated and down-regulated, respectively. In the 250 ng/ml-treated group, 195 and 152 genes were up- and down-regulated more than two-fold, respectively. However, it should be noted that a concentration as low as 10 ng/ml Wnt3A is capable of inducing significant changes in gene expression.

There is a strong correlation in the behavior of the genes in the 24-hour Wnt3A-treated groups. Genes up- (or down-) regulated in one of these groups were also up- (or down-) regulated in the other two groups. Interestingly, there exists an opposite correlation between the genes up- or down-regulated in the four-hour-treated group compared to the genes in the 24-hour-treated group. In other words, genes up-regulated in the four-hour-treated group were down-regulated in the 24-hour-treated group and visa versa.

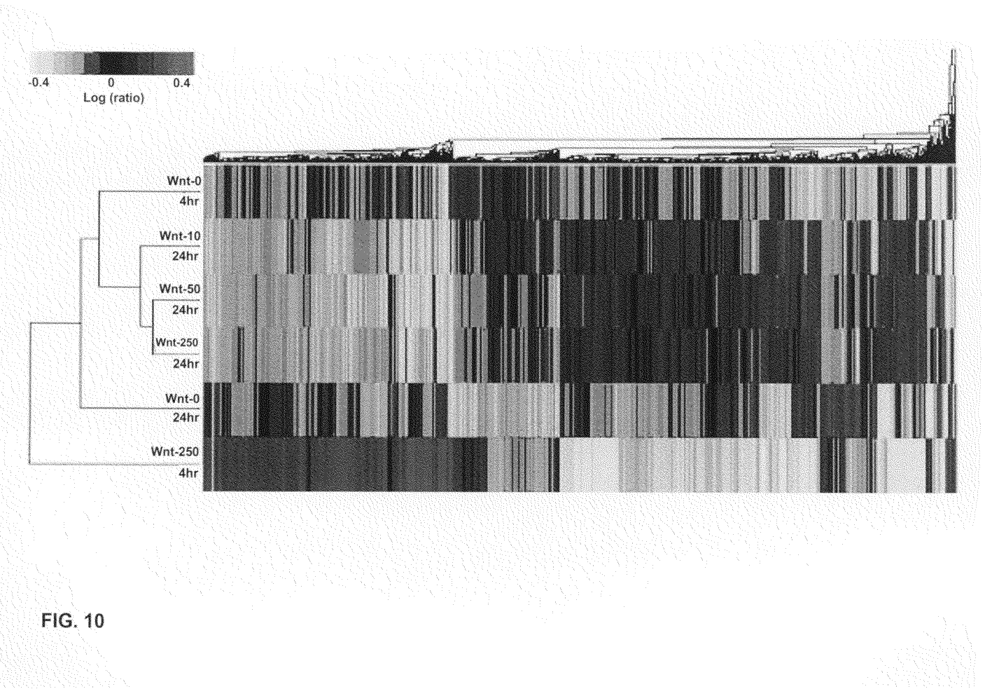

Cluster analysis of the expression profiles of genes significantly different in one of the six groups (4500 genes), in which genes with a comparable expression level are grouped, clearly shows that the three groups treated with 10, 50 and 250 ng/ml Wnt3A during 24 hours, respond in a similar way (FIG. 10). Importantly, the cluster analysis of 4500 significantly changed genes (p<0.000001) shows that the expression profiles of these three 24-hour Wnt3A-treated lungs are strongly related to the four-hour-untreated lungs and these four conditions resemble each other more than the 24-hour-untreated lungs. This shows that the transcriptome of 24-hour-cultured lungs remains younger when cultured in the presence of Wnt3A. In other words, Wnt3A inhibits, at least partially, the differentiation of fetal lung tissue.

The cluster analysis also shows that the four-hour Wnt3A-treated group has the most different expression profile of all six groups. Furthermore, it shows that this profile is for the majority of genes (circa 75%) opposite from the profiles of the 24-hour Wnt3A-treated groups. Apparently, a strong feedback mechanism occurs upon Wnt3A stimulation after 24 hours, a mechanism which ultimately results in the inhibition of the differentiation and development of the lung.

### CONCLUSIONS

In this example, we have shown that the addition of Wnt3A induces the nuclear accumulation of beta-catenin in primordial lung tissue, the hallmark of wnt-signal transduction. In addition, we show that Wnt3A administration induces the expression of a wnt-reporter construct transfected in fetal lung tissue, at least between 10 and 1000 ng/ml Wnt3A. The primordial lung thus has all the necessary components of the wnt-signal transduction pathway to recognize the Wnt3a polypeptide and to transduce its signal intracellularly.

The addition of Wnt3a has a profound effect upon fetal lung development. Differentiation, cell proliferation and apoptosis are greatly altered upon addition of Wnt3a to the culture medium. The effective concentration depends on the cell type and the cellular function.

Using morphological criteria, marker gene analysis and microarray analysis, we show that differentiation of the primordial lung epithelium is effectively inhibited between a concentration of 10 and 2000 ng/ml Wnt3A, with an optimum between 50 and 500 ng/ml.

Proliferation of the bronchial and alveolar epithelium is markedly enhanced between 30 and 500 ng Wnt3a/ml culture medium, whereas at concentrations above 2000 ng/ml, proliferation is inhibited.

Apoptosis in the mesenchyme is induced at concentrations greater than 100 ng/ml, whereas apoptosis in the epithelium is induced at concentrations higher than 1000 ng/ml. The mesenchyme is thus much more sensitive for Wnt3A-induced apoptosis than the epithelium.

The effects of Wnt3a upon differentiation, proliferation and apoptosis are strongly dependent upon cell type and dosage. Our results show that at certain concentrations, Wnt3a can, at the same time, inhibit alveolar differentiation, enhance epithelial proliferation and induce apoptosis in mesenchymal cells. The range in which all these three events occur in the above-described hanging drop culture system is between 100 and 1000 ng Wnt3a/ml culture medium. The addition of Wnt3a is, therefore, suitable for selectively culturing and expanding in total number the undifferentiated stem cell population of the lung and for selectively inducing apoptosis in a mesenchymal cell population.

In addition, we have shown that Wnt3A not only inhibits differentiation, it also restricts differentiation along the proximal-distal axis of the lung. This biological effect is also dosage-dependent upon the concentration Wnt3A. As a rule of thumb, the higher the concentration of Wnt3A, the more the differentiation is restricted to tissue types present in the proximal part of the lung, i.e., the tracheal epithelium with surrounding mesenchyme and cartilage. The concentration range in which this biological effect took place is in the range of 10 to 1000 ng/ml. Between 10 and 250 ng/ml, Wnt3A differentiation of distal epithelium was suppressed, whereas between 250 and 1000 ng/ml, Wnt3a differentiation became primarily restricted to proximal lung tissue, such as bronchial epithelium, tracheal epithelium and cartilage. The addition of a certain concentration of Wnt3A polypeptide is thus useful for selectively differentiating primordial lung cells into proximal lung tissue. This tissue, on its turn, is then suitable for treating diseases or injuries of the upper airways.

### REFERENCES

Behrens J., J. P. von Kries, M. Kuhl, L. Bruhn, D. Wedlich, R. Grosschedl and W. Birchmeier, 1996. Functional interaction of beta-catenin with the transcription factor LEF-1. Nature 382:638-42.
Bhanot P., M. Brink, C. H. Samos, J. C. Hsieh, Y. Wang, J. P. Macke, D. Andrew, Cabilly et al., U.S. Pat. No. 4,816,567.
Nathans J. and R. Nusse, 1996. A new member of the frizzled family from Drosophila functions as a wingless receptor. Nature 382:225-30.
Bienz M. and H. Clevers. Linking colorectal cancer to Wnt signaling. Cell 2000 Oct. 13; 103(2):311-20.
Brown J. D., S. E. Hallagan, L. L. McGrew, J. R. Miller and R. T. Moo, 2000. The maternal Xenopus beta-catenin signaling pathway, activated by frizzled homologs, induces goosecoid in a cell non-autonomous manner. Dev. Growth Differ. 42:347-57, 2000.
Clackson T., H. R. Hoogenboom, A. D. Griffiths and G. Winter. Making antibody fragments using phage display libraries. Nature 1991 Aug. 15; 352(6336):624-8.
Hinck L., W. J. Nelson and J. Papkoff, 1994. Wnt-1 modulates cell-cell adhesion in mammalian cells by stabilizing beta-catenin binding to the cell adhesion protein cadherin. J. Cell Biol. 124:729-41.
Kielman M. F., M. Rindapää, C. Gaspar, N. van Poppel, C. Breukel, S. van Leeuwen, M. M.
Taketo, S. Roberts, R. Smits, R. Fodde, 2002. Apc modulates embryonic stem-cell differentiation by controlling the dosage of Beta catenin signaling. Nature Genetics 32:594-605.
Kohler G. and C. Milstein. Continuous cultures of fused cells secreting antibody of predefined specificity. Nature 1975 Aug. 7; 256(5517):495-7.
Korinek V., N. Barker, K. Willert, M. Molenaar, J. Roose, G. Wagenaar, M. Markman, W. Lamers, O. Destree and H. Clevers, 1998. Two members of the Tcf family implicated in Wnt/beta-catenin signaling during embryogenesis in the mouse. Mol. Cell Biol. 18:1248-56.
Lee S. M., S. Tole, E. Grove and A. P. McMahon, 2000. A local Wnt-3a signal is required for development of the mammalian hippocampus. Development 127:457-67.
Marks J. D., H. R. Hoogenboom, T. P. Bonnert, J. McCafferty, A. D. Griffiths, G. Winter. By-passing immunization. Human antibodies from V-gene libraries displayed on phage. J. Mol. Biol. 1991 Dec. 5; 222(3):581-97.
McMahon A. P., B. J. Gavin, B. Parr, A. Bradley and J. A. McMahon, 1992. The Wnt family of cell signaling molecules in postimplantation development of the mouse. Ciba Found. Symp. 165:199-212; discussion 212-8.
Matthews W. and T. W. Austin, 1997. Uses of Wnt polypeptides. U.S. Pat. No. 6,159,462.
Morrison S. L., M. J. Johnson, L. A. Herzenberg, and V. T. Oi. Chimeric human antibody molecules: mouse antigen-binding domains with human constant region domains. Proc. Natl. Acad. Sci. U.S.A. 1984 November; 81(21):6851-6855.
Nusse R. and H. E. Varmus, 1992. Wnt genes. Cell 69:1073-87.
Reya T., A. W. Duncan, L. Ailles, J. Domen, D. C. Scherer, K. Willert, L. Hintz, R. Nusse, I. L.
Weissmann. A role for Wnt signaling in self-renewal of haematopoietic stem cells. Nature May 2003, 409-414.
Stark K., S. Vainio, G. Vassileva and A. P. McMahon, 1994. Epithelial transformation of metanephric mesenchyme in the developing kidney regulated by Wnt-4. Nature 372:679-83.
Willert K. and R. Nusse, 1998. Beta-catenin: a key mediator of Wnt signaling. Curr. Opin. Genet. Dev. 8:95-102.
Willert K., J. D. Brown, E. Danenberg, A. W. Duncan, I. L. Weissman, T. Reya, J. R. Yates 3rd, and R. Nusse. Wnt proteins are lipid-modified and can act as stem cell growth factors. Nature 2003 May 22; 423(6938):448-52.
Wodarz A. and R. Nusse, 1998. Mechanisms of Wnt signaling in development. Annu. Rev. Cell Dev. Biol. 14:59-88.

1. A method for inhibiting differentiation of a lung stem cell, said method comprising:
providing said lung stem cell in vitro, in a tissue culture, with means for up-regulating a Wnt-pathway.

2. The method according to claim 1, wherein the inhibition of differentiation of said lung stem cell induces the lung stem cell to proliferate.

3. The method according to claim 1, wherein said means for up-regulating a Wnt-pathway comprises a soluble Wnt3a factor or a functional part, derivative or analogue of Wnt3a factor that up-regulates a Wnt-pathway.

4. The method according to claim 1, wherein said lung stem cell is provided with means for up-regulating a Wnt-pathway in an amount of at least 1 ng and at most 2000 ng per ml tissue culture fluid.

5. The method according to claim 4, wherein said lung cell is provided with means for up-regulating a Wnt-pathway in an amount is from at least 10 ng to at most 1500 ng per ml tissue culture fluid.

6. The method according to claim 5 wherein the amount of means for up-regulating a Wnt-pathway is from at least 20 ng to at most 1000 ng per ml tissue culture fluid.

7. The method according to claim 6 wherein the amount of means for up-regulating a Wnt-pathway is from at least 30 ng to at most 500 ng per ml tissue culture fluid.

8. A method for enriching a population of stem cells in a population of mesenchymal cells comprising said stem cells, said method comprising:
inducing apoptosis in the mesenchymal cells by providing said mesenchymal cells with means for up-regulating a Wnt-pathway.

9. The method according to claim 8, wherein the stem cells comprise lung stem cells.

10. The method according to claim 8, wherein said means for up-regulating the Wnt-pathway comprise a soluble Wnt3a factor or a functional part, derivative or analogue of a soluble Wnt3a factor that up-regulates the Wnt-pathway.

11. The method according to claim 8, wherein said mesenchymal cells are provided with means for up-regulating the Wnt-pathway in an amount of at least 50 ng and at most 1500 ng per ml tissue culture fluid containing said population of mesenchymal cells.

12. The method according to claim 11, wherein said mesenchymal cells are provided with means for up-regulating the Wnt-pathway in an amount of at least 100 ng and at most 1000 ng per ml tissue culture fluid containing said population of mesenchymal cells.

13. A method for obtaining a plurality of stem cells from a population of mesenchymal cells and stem cells, said method comprising:
providing the mesenchymal cells with an apoptosis inducing amount of a Wnt-pathway up-regulating substance, and providing the stem cells with a differentiation inhibiting amount of a Wnt-pathway up-regulating substance,
so as to obtain a plurality of stem cells from a population.

14. The method according to claim 13, wherein said stem cells are lung stem cells.

15. The method according to claim 13, wherein said Wnt-pathway up-regulating substance comprises a soluble Wnt3a factor or a functional part, derivative or analogue of a soluble Wnt3a factor that up-regulates the Wnt-pathway.

16. The method according to claim 13, wherein said Wnt-pathway up-regulating substance is provided in an amount of at least 50 ng and at most 1500 ng per ml tissue culture fluid containing said population of mesenchymal cells and stem cells.

17. The method according to claim 13, wherein differentiation of a distal lung cell type is at least partly inhibited by administering 10-250 ng Wnt3a or a functional part, derivative or analogue thereof per ml tissue culture fluid containing said population of mesenchymal cells and stem cells.

18. The method according to claim 17, wherein said distal lung cell type is a type I or type II alveolar cell.

19. The method according to claim 13, wherein proliferation and differentiation of an upper airway cell type is selectively increased by administering 10-750 ng Wnt3a or a functional part, derivative or analogue thereof /ml tissue culture fluid containing said population of mesenchymal cells and stem cells.

20. The method according to claim 19 wherein said upper airway cell type is a tracheal and/or bronchial epithelial cell.

21. A differentiated cell obtained by the method according to claim 19.

22. A kit comprising:
a stem cell culture medium and
a soluble Wnt3a factor or a functional part, derivative or analogue thereof in an amount of at least 1 to at most 2000 ng per ml stem cell culture medium.
